(19) United States
(12) Patent Application Publication
(10) Pub. No.: US 2022/0030896 A2
(43) Pub. Date: Feb. 3, 2022

(54) CHOLINE ESTER-CONTAINING COMPOSITION FOR ORAL INGESTION

(71) Applicant: Shinshu University, Matsumoto-shi, Nagano (JP)

(72) Inventor: KOZO NAKAMURA, Kamiina-gun, Nagano (JP)

(73) Assignee: Shinshu University, Matsumoto-shi, Nagano (JP)

(21) Appl. No.: 16/341,748

(22) PCT Filed: Oct. 16, 2017
(86) PCT No.: PCT/JP2017/037389

§ 371 Date: Apr. 12, 2019

(30) Foreign Application Priority Data
Oct. 14, 2016 (JP) 2016-202909

(51) Int. Cl. A23C 3/02; A23C 9/123

(52) U.S. Cl. CPC A23C 3/02; A23C 9/123

(57) ABSTRACT

The object of the present invention is to provide a composition having a blood pressure-lowering effect and/or an anti-stress effect, wherein an active ingredient of a choline ester such as acetylcholine, and the like, can be easily administered to humans by the oral pathway.

Consequently, the object of the present invention is to provide a novel composition for oral ingestion having a blood pressure-lowering effect and a vasodilatory effect, wherein a choline ester such as acetylcholine, and the like, can be easily administered to humans by the oral pathway, and to provide food which is a useful supply source therefore.

### TECHNICAL FIELD

The present invention relates to a composition having a blood pressure-lowering effect and/or an anti-stress effect and comprising a choline ester, which is a compound wherein a choline and an organic acid are ester-bonded, as an active ingredient, and a production method thereof.

### BACKGROUND ART

Among choline esters, acetylcholine is known to be an essential substance for life activity as a neurotransmitter in mammals. Moreover, in 1929, a blood pressure-lowering substance different from histamine was isolated from the spleen of a horse; this functional substance was chemically identified as acetylcholine (Non-Patent Document 1). Furthermore, a blood pressure-lowering substance in ergot has been revealed to be acetylcholine, and it has been confirmed that fungi produce acetylcholine (Non-Patent Document 2). Acetylcholine is contained in edible plants, edible fungi, royal jelly, milk, and the like, and acetylcholine is also present in Bacillus subtilis and yeasts; it has also been reported that in edible plants the acetylcholine content in eggplants and bamboo shoots is high (Patent Documents 3 to 7).

In addition to acetylcholine, a plurality of choline esters has also been discovered.

In 1953, propionylcholine, which has a propionyl group wherein the carbon chain is one carbon longer than in an acetyl group, was discovered in ox spleen (Non-Patent Documents 8 and 9). Thereafter, in addition to bull spermatozoa, haemolymphs and smooth muscles of the European crayfish, the American horseshoe crab, common European cockles, soft-shell clams, Mediterranean mussels, swan mussels (Anadonta cygnea) and escargots de Bourgogne (Helix pomatia), and the electricity generating tissue culture of Torpedo, propionylcholine production was confirmed in garden crotons (Codiaeum veriegatum), mung beans (Vigna radiata), Chinese plantain, poplar, birch, and the like (Non-Patent Documents 10 to 13).

Butyrylcholine was discovered in 1954 from a brain extract (Non-Patent Document 14), and has been shown to be present in arthropods and molluscs together with acetylcholine and propionylcholine (Non-Patent Document 11).

In addition to propionylcholine and butyrylcholine, a number of choline esters have also been confirmed from molluscs. For example, the structure of urocanoyl choline has been determined from one type of Muricidae, the structure of ββ-dimethyl acroyl choline (senecioyl choline) has been determined from one type of Reishia (Thais) bronni, the structure of acroyl choline has been determined from one type of Buccinidae, and the structure of imidazole propionylcholine has been determined from one type of Reishia (Thais) bronni (Non-Patent Documents 15-18).

Having studied the blood pressure-lowering and vasodilatory active ingredients contained in fermented buckwheat (lactic acid-fermentation product of buckwheat plants), the present inventor has provided an extract composition comprising a plurality of choline ester-based quaternary alkylammonium compounds containing at least acetylcholine and propionylcholine; the present inventor has further shown that when purified acetylcholine, propionylcholine and butyrylcholine were fed to spontaneously hypertensive rats (SHR) by single oral administration, a blood pressure-lowering effect is obtained (Patent Documents 1, 2). On the other hand, as is clear from the Interview Form (Non-Patent Document 19) of medicine comprising acetylcholine chloride as an active ingredient, which states that “when acetylcholine is orally administered, it is decomposed in the gastrointestinal tract and hardly absorbed, therefore it was injected,” choline esters are conventionally administered to humans by injection and the effect due to oral ingestion has not been investigated.

### CITATION LIST

### Patent Documents

[Patent Document 1] WO 2015-147251
[Patent Document 2] JP A 2015-189745
[Patent Document 3] JP A Hei 6-065068

### Non-Patent Documents

[Non-Patent Document 1] Dale H H, Dudley H W. The presence of histamine and acetylcholine in the spleen of the ox and the horse. J Physiol 68:97-123, 1929.
[Non-Patent Document 2] Ewins A J. Acetylcholine, a new active principle of ergot. Biochem J 8:44-49, 1914.
[Non-Patent Document 3] Yoshie Momonoki: Acetylcholine in plants, Chemical Regulation in Plants 30 (1), 49-61 (1995) (In Japanese).
[Non-Patent Document 4] Koichiro Kawashima: Roots of acetylcholine and non-neuronal acetylcholine, Basic Aging Research 34 (4), 12-24 (2010) (In Japanese).
[Non-Patent Document 5] Masato Shinoda et al.: Blood flow-increasing factor in royal jelly, Pharmaceutical Journal 98 (2), 139-145 (1978) (In Japanese).
[Non-Patent Document 6] Whittaker V P. Acetylcholine in Milk. Nature 181:856-857, 1958.
[Non-Patent Document 7] Horiuchi Y, Kimura R, Kato N, Fujii T, Seki M, Endo T, Kato T, Kawashima, K. Evolutional study on acetylcholine expression. Life Sci 72:1745-1756, 2003.
[Non-Patent Document 8] Banister J, Whittaker V P, Wijesundera S. The occurrence of homologues of acetylcholine in ox spleen. J Physiol 121(1):55-71, 1953.
[Non-Patent Document 9] Gardiner J E, Whittaker V P. The identification of propionylcholine as a constituent of ox spleen. Biochem J 58(1):24-29, 1954.
[Non-Patent Document 10] Bishop M R, Sastry B V, Stavinoha W B. Identification of acetylcholine and propionylcholine in bull spermatozoa by integrated pyrolysis, gas chromatography and mass spectrometry. Biochim Biophys Acta 500(2):440-444, 1977.
[Non-Patent Document 11] Wolfgang W, Jutta N, Dettmar W. Distribution of cholinesters and cholinesterases in haemolymphs and smooth muscles of molluscs. Comp Biochem Phys C 61(1):121-131, 1978.
[Non-Patent Document 12] O'Regan S. The synthesis, storage, and release of propionylcholine by the electric organ of Torpedo marmorata. J Neurochem 39(3):764-772, 1982.
[Non-Patent Document 13] Miural G A, Shin T M. Identification of proprionylcholine in higher plants. Physiol Plant 62:341-343, 1984.
[Non-Patent Document 14] Holtz P, Schumann H J. Butyrylcholine in brain extracts. Naturwissenschaften 41:306, 1954.
[Non-Patent Document 15] Erspamer V, Benati O. Identification of murexine as beta-[imidazolyl-(4)]acrylcholine. Science 117:161-162, 1953.
[Non-Patent Document 16] Keyl M J, Michaelson I A, Whittaker V P. Physiologically active choline esters in certain marine gastropods and other invertebrates. J Physiol 139:434, 1957.
[Non-Patent Document 17] Whittaker V P. Acrylylcholine: a new naturally occurring pharmacologically active choline ester from Buccinum undatum. Biochem Pharmacol 1(4):342-346, 1959.
[Non-Patent Document 18] Roseghini M. Occurrence of dihydromurexine (imidazole propionylcholine) in the hypobranchial gland of Thais (purpura) haemastoma. Experientia 27(9):1008-1009, 1971.
[Non Patent Document 19] Interview Form for OVISOT® for injection 0.1 g, January 2013
[Non-Patent Document 20] The latest basic pharmacology (Supervision: Keijiro Takagi, Tsutomu Kameyama, Editing: Sachiko Oishi, Susumu Okabe, Issuer: Hirokawa Shoten, page 96, lines 6 to 10 (in Japanese).
[Non-Patent Document 21] Abstracts of the seminar of the 2016 conference of the Japan Society of Agricultural Chemistry, 4E 076, published on Mar. 5, 2016.
[Non-Patent Document 22] Kleiber, M. The fire of life. An introduction to animal energetics. New York: Wiley, 1961.
[Non-Patent Document 23] Kim J M, Lee S W, Kim K M, Chang U J, Song J C, Suh H J. Anti-stress effect and functionality of yeast hydrolysate SCP-20. Europe Food Res Technol 217(2):168-172, 2003.
[Non-Patent Document 24] Armando I, Carranza A, Nishimura Y, Hoe K L, Barontini M, Terron J A, Falcon-Neri A, Ito T, Juorio A V, Saavedra J M. Peripheral administration of an angiotensin II AT(1) receptor antagonist decreases the hypothalamic-pituitary-adrenal response to isolation Stress. Endocrinology 142(9):3880-3889, 2001.
[Non-Patent Document 25] Nakamura K, Okitsu S, Ishida R, Tian S, Igari N, Amano Y. Identification of natural lactoylcholine in lactic acid bacteria-fermented food. Food Chem 201:185-189, 2016.

### SUMMARY OF INVENTION

### Problems to be Solved by Invention

The inventor of the present invention has focused on the fact that, if the use of choline ester by oral ingestion in humans becomes possible, even though blood pressure can be lowered safely and easily without any negative effect on the body, a technology for suitably administering acetylcholine to humans via the oral pathway and a supply source for said technology has not yet been discovered, and therefore considers that it is important to clarify these points.

Consequently, the object of the present invention is to provide a novel composition for oral ingestion having a blood pressure-lowering effect and a vasodilatory effect, wherein an active ingredient of a choline ester such as acetylcholine, and the like, can be easily administered to humans via oral pathway, and to provide food which is a useful supply source therefore.

### Means of Solving Problems

In the course of the extensive studies undertaken to solve the above problems, the inventor of the present invention, despite the conventional view that acetylcholine up to now cannot be orally administered to humans, has surprisingly found that there is a proper dosage for choline esters to exhibit a blood pressure-lowering effect and a vasodilatory effect by oral administration and that, moreover, said dosage has an anti-stress effect; the inventor of the present invention has moreover found edible plants suitable as source for efficiently supplying choline esters for this purpose and, as a result of further studies, has completed the present invention.

Consequently the present invention relates to:

[1] a composition having a blood pressure-lowering effect and an anti-stress effect and comprising a choline ester as an active ingredient, wherein the composition is for oral ingestion and the choline esters content is 5 μg to 50 mg.
[2] the composition according to [1] above, wherein the composition is a food composition.
[3] the composition according to [1] above, wherein the composition is a blood-pressure-lowering and/or anti-stress pharmaceutical composition.
[4] the composition according to any one of [1] to [3] above, wherein the choline ester is derived from an edible plant.
[5] the composition according to any one of [1] to [4] above, consisting of a lyophilized powder and/or a hot air-dried powder of edible plants.
[6] the composition according to [5] above, consisting a lyophilized powder and/or a hot air-dried powder of edible plants, which can pass through a 20 mesh sieve.
[7] the composition according to any one of [1] to [4] above, wherein the composition is an extract obtained by extracting edible plants with ethanol or hydrous ethanol.
[8] the composition according to any one of [4] to [7] above, wherein the edible plant is a fruit of a Solanaceae Solanum eggplant species (Solanum melongena) and/or a young shoot of a bamboo subfamily of Piceaceae (Poaceae, Bambusoideae, Bambuseae).
[9] the composition according to any one of [1] to [3] above, wherein the choline ester comprises one or more selected from the group consisting of acetylcholine, butyrylcholine and propionylcholine.
[10] the composition according to [9] above, wherein the choline ester does not contain lactoylcholine.
[11] the composition according to any one of [1] to [10] above, wherein the concentration of choline ester is 5 μg/g to 250 mg/g and the intake amount per day is adjusted from 5 μg to 50 mg.
[12] the composition according to any one of [1] to [11] above, wherein the composition is frozen.
[13] the composition according to any one of [1] to [3] above, wherein the composition is a part or the whole of the frozen fruit of a Solanaceae Solanum eggplant species (Solanum melongena).
[14] a method of producing a composition for oral ingestion having a blood pressure-lowering effect and/or an anti-stress effect and comprising a choline ester as an active ingredient, wherein the method comprises a step for making an edible plant into a lyophilized powder and/or a hot air-dried powder and a step for dispensing the lyophilized powder and/or hot air-dried powder so that the choline ester content is 5 μg to 50 mg.
[15] the method according to [14] above, wherein the edible plant is a fruit of a Solanaceae Solanum eggplant species (Solanum melongena) and/or a young shoot of a bamboo subfamily of Piceaceae (Poaceae, Bambusoideae, Bambuseae).
[16] the method according to [14] or [15] above, wherein the method further comprises a step for heating the edible plant.
[17] the method according to any one of [14] to [16] above, wherein the method further comprises a step for suspending the lyophilized powder and/or hot air-dried powder of an edible plant in water, and a step for adding an acid to the suspension obtained.
[18] the method according to [17], wherein the method further comprises a step for adjusting the suspension to which acid has been added to a pH of 5.5 to 4.5.
[19] a composition for oral ingestion having a blood pressure-lowering effect and/or an anti-stress effect and comprising a choline ester as an active ingredient, which is produced by a method according to any one of [14] to [18] above.
[20] a method for producing an extract for oral ingestion having a blood pressure-lowering effect and/or an anti-stress effect and comprising a choline ester as an active ingredient, wherein the method comprises a step for extracting an edible plant or a lyophilized powder and/or a hot air-dried powder of an edible plant with ethanol or hydrous ethanol.
[21] the method according to [20] above, wherein the method comprises a step for extracting an edible plant or a lyophilized powder and/or a hot air-dried powder of an edible plant with ethanol.
[22] the method according to 20 above, wherein the method comprises a step for extracting an edible plant or a lyophilized powder and/or a hot air-dried powder of an edible plant with hydrous ethanol, and the ethanol concentration of the hydrous ethanol is 25 to 60% (w/w), or 95% (w/w) or more.
[23] the method according to any one of [20] to [22] above, wherein L-ascorbic acid is added to the ethanol or the hydrous ethanol used for the extraction.
[24] the method according to any one [20] to [23] above, wherein the method comprises a step for adjusting the choline ester content of the extract from 5 μg to 50 mg.
[25] an extract for oral ingestion having a blood pressure-lowering effect and/or an anti-stress effect and comprising a choline ester as an active ingredient produced according to any one of [20] to [24] above.

### Advantageous Effects of Invention

The present invention provides a composition which is easy to ingest over a long period of time and which has a blood pressure-lowering effect even when orally ingesting choline esters as an active ingredient without injection. In particular, in the case of a composition comprising a lyophilized powder of an edible plant, it can be processed at a very low cost, since it does not require steps for extraction or purification. Furthermore, in order to increase and sterilize the choline esters, it is possible to heat the powder at certain conditions before lyophilisation, and to add an acid before or after lyophilisation in order to increase the stability of the choline ester. Since there is abundant experience of the use of choline esters as food, the composition of the present invention is highly safe and can be used as a food composition or as a pharmaceutical composition for lowering blood pressure.

Furthermore, according to the composition of the present invention, it is possible to use foods, beverages and medicines in various embodiments such as a product of an edible plant (fresh plant) cut into pieces so as to comprise a predetermined amount of choline ester, a product of said edible plant that has been heated and frozen, a dried powder of an edible plant (lyophilized powder, hot air-dried powder), a suspension of said dried powder, an extract of choline esters extracted from fresh plants, a concentrated extract of said extract, and the like.

Choline ester acts on cholinergic receptors (muscarinic acetylcholine receptors and nicotinic acetylcholine receptors), but for stimulating the nicotinic acetylcholine receptors it is necessary to use acetylcholine at a higher concentration than for muscarinic acetylcholine receptor stimulation (Non-Patent Document 20). In addition, blood pressure is reduced due to the activity of the muscarinic acetylcholine receptor, but such an activity is thought to come about because the sympathetic nerve activity becomes dominant due to the activity of the nicotine acetylcholine receptor and because the activity of the muscarinic acetylcholine receptor is cancelled. The present invention is based on the finding that when choline ester is orally ingested in an extremely small amount, it exhibits an activity that is different from when it is orally ingested in large amounts. In other words, compared to the 0.1 g of an Ovisot (registered trademark) injection, the choline ester amount in the composition of the present invention is extremely small, but it sufficiently acts on the muscarinic acetylcholine receptor and exhibits a blood pressure-lowering effect; moreover, it does not act on the nicotinic acetylcholine receptor, and it is an amount that does not cancel the blood pressure lowering effect. Since the choline ester content corresponds to the balance between the activities of the two receptors, the composition of the present invention, contrary to the common technical knowledge that acetylcholine cannot be orally administered, exhibits a blood pressure-lowering effect by oral ingestion.

In the present invention, when edible plants having a high choline ester content such as eggplants and bamboo shoots are used, oral use is preferred because a small amount of the plants is sufficient. For example, when choosing a plant with low choline ester content such as lettuce, it is necessary to ingest about 7.5 kg or more per day in terms of fresh weight, which is not realistic as a daily dose of ingestion. Even when lettuce is lyophilized, the yield is about 3.60%, and it is desirable to ingest about 270 g/day. On the other hand, for eggplants, the estimated daily intake obtained from animal test results using SHR was only 0.41 g in terms of fresh weight; therefore, it is possible to obtain a blood pressure-lowering effect at an amount that can be continually ingested on a daily basis. Moreover, when eggplants are used as they are or when they are heated and lyophilized, the recommended daily intake can be further reduced.

Consequently, it can be made into a very sensible and economical food for oral ingestion even when it is used as processed food.

### BRIEF DESCRIPTION OF DRAWINGS

FIG. 1 is a graph showing the yield after lyophilisation of different types of fresh agricultural products.

FIG. 2 is a graph showing the choline ester and choline content of different types of fresh agricultural products. The vertical axis has a logarithmic scale. AcCh: acetylcholine, BuCh: butyrylcholine, PrCh: propionylcholine, Ch: choline.

FIG. 3 is a graph showing the change over time of choline ester content and choline content in fresh eggplants during 1 to 5 days of storage at ambient temperature (p*<0.05, p**<0.01 vs First day of the course). AcCh: acetylcholine, BuCh: butyrylcholine, PrCh: propionylcholine, Ch: choline.

FIG. 4 is a graph showing the choline ester content in fresh eggplants at ambient temperature and after 6 days of refrigerated storage. (P*<0.05, p**<0.01). The vertical axis has a logarithmic scale. AcCh: acetylcholine, BuCh: butyrylcholine, PrCh: propionylcholine.

FIG. 5 is a graph showing the change over time in the amount of acetylcholine with respect to pH when a standard sample is used. AcCh: acetylcholine.

FIG. 6 is a graph showing the change over time in the amount of acetylcholine with respect to pH when an eggplant extract is used. AcCh: acetylcholine.

FIG. 7 is a graph showing the change in the amount of acetylcholine in eggplants due to heating. AcCh: acetylcholine.

FIG. 8 is a graph showing the results of single oral administration tests (systolic blood pressure) of lyophilized eggplant powder containing 1.00×10⁻⁹ mol/kg equivalent of acetylcholine in SHR (p*<0.05 vs pure water administration).

FIG. 9 is a graph showing the results of single oral administration tests (diastolic blood pressure) of lyophilized eggplant powder containing 1.00×10⁻⁹ mol/kg equivalent of acetylcholine in SHR.

FIG. 10 is a graph showing the results of single oral administration tests (heart rate) of lyophilized eggplant powder containing 1.00×10−9 mol/kg equivalent of acetylcholine in SHR.

FIG. 11 is a graph showing the results of single oral administration test (systolic blood pressure) of lyophilized eggplant powder in WKY rats (p*<0.05, p**<0.01 vs pure water administration).

FIG. 12 is a graph showing the vascular isometric tension test results of lyophilized eggplant powder (p*<0.05, p**<0.01 vs lyophilized 20 mesh-pass; p #<0 0.05, p ##<0.01 vs hot air drying 20 mesh pass).

FIG. 13 is a graph showing the results of repeated oral administration tests (systolic blood pressure) of lyophilized eggplant powder in SHR (p*<0.05, p**<0.01 vs pure water administration).

FIG. 14 is a graph showing the results of repeated oral administration tests (diastolic blood pressure) of lyophilized eggplant powder in SHR (p*<0.05 vs pure water administration).

FIG. 15 is a graph showing the results of repeated oral administration tests (urinary adrenaline content change) of lyophilized eggplant powder in SHR (p*<0.05, p**<0.01 vs pure water administration).

FIG. 16 is a graph showing the result of repeated oral administration tests (urinary noradrenaline content change) of lyophilized eggplant powder in SHR (p*<0.05, p**<0.01 vs pure water administration).

### EMBODIMENTS FOR CARRYING OUT INVENTION

The present invention relates to a composition having a blood pressure-lowering effect and a choline ester as an active ingredient and further relates to said composition having an anti-stress effect.

The composition according to the present invention can be orally ingested and can have a choline ester content of 5 μg to 50 mg.

In the composition according to the present invention, the choline ester content is 5 to 500 μg, preferably 5 to 250 μg, more preferably 10 to 50 μg, and particularly preferably 25 μg. Such a choline ester content is preferable, for example, when it is used for subjects with hypertension.

Moreover, the choline ester content may be 5 μg to 50 mg, preferably 500 μg to 50 mg, and more preferably 500 μg to 5 mg. Such a choline ester content is preferable, for example, when it is used for a healthy subject that has high blood pressure (not hypertension).

The composition according to the present invention may be a food composition or a pharmaceutical composition. When the composition according to the present invention is a pharmaceutical composition, it may be a pharmaceutical composition for lowering blood pressure and/or for reducing stress.

The choline esters, which are the active ingredients of the composition according to the present invention, may be derived from animals, plants and microorganisms; however, choline esters derived from organisms that have been used for human food are preferred, in particular, choline esters derived from edible plants are preferred.

The edible plants are not particularly limited as long as they contain a choline ester. Examples of edible plants include cucumbers, tomatoes, paprika, green peppers, eggplants, asparagus, Japanese mountain yams, cabbages, lettuce, carrots, apples, Shishito peppers (sweet species of Capsicum annuum), Japanese Nashi pears, grapes, Daikon sprouts, broccoli, alfalfa, soybeans, buckwheat, bamboo shoots, and the like. From the point of view of the acetylcholine content, Solanaceae Solanum eggplant species (Solanum melongena) and/or a bamboo subfamily of Piceaceae (Poaceae, Bambusoideae, Bambuseae) are preferred, in particular the fruit of a Solanaceae Solanum eggplant species (Solanum melongena) and/or the young shoot of a bamboo subfamily of Piceaceae (Poaceae, Bambusoideae, Bambuseae) are preferred.

Among the varieties of Solanaceae Solanum eggplant species (Solanum melongena), it is preferred to use ‘Senshu’ water eggplants, ‘Batten’ eggplants, ‘Koryo’ salad eggplants (also called ‘Binan’ eggplants), ‘Higomurasaki’ eggplants, ‘Oonaga (very long) eggplants, ‘Chikuyo’ eggplants, and the like; ‘Senshu’ water eggplants, ‘Batten’ eggplants, ‘Koryo’ salad eggplants and ‘Higomurasaki’ eggplants are preferred because they can be consumed as fresh produce. ‘Higomurasaki eggplants are particularly preferred.

Examples of choline esters that can be included in the composition according to the present invention include acetylcholine, butyrylcholine, propionylcholine, lactoylcholine, and the like; wherein the composition may contain one or more types of these choline esters. In particular, when the choline ester is derived from a plant, the composition according to the present invention contains one or more selected from the group consisting of acetylcholine, butyrylcholine and propionylcholine, and does not contain lactoylcholine.

In the composition according to the present invention, the daily intake of choline ester is adjusted within a predetermined range.

For example, the choline ester content in one sachet may be adjusted within the range of 5 to 125 μg, preferably 10 to 75 μg, and particularly preferably 15 to 50 μg, which can be orally administered one to several times (preferably about 3 times) a day. Moreover, the total amount of choline esters may, for example, be adjusted so as to be within the above range by a plurality of sachets. In this case, the choline ester concentration is 5 to 2500 μg/g.

Furthermore, when for example the daily intake of choline ester is adjusted to a figure larger than the above numerical range, in other words, when it is adjusted from 5 μg to 50 mg, preferably 500 μg to 12.5 mg, more preferably 500 μg to 1.25 mg, it is possible to increase the amount of choline ester per sachet according to the above method. In this case, the choline ester concentration may be 500 to 250 mg/g.

The composition according to the present invention is adjusted to a predetermined choline ester content. In the composition according to the present invention, it is possible to use, for example, cut up fresh agricultural products, frozen products, lyophilized products, extracts, and the like, by adjusting the choline ester content. The composition according to the present invention is preferably a composition comprising lyophilized powder and/or an extract of edible plants.

The composition according to the present invention may be prepared by dividing it into daily doses so as to adjust the ester content to the daily intake, and each dose may be packed in a vacuum pack, or the like, to prevent deterioration of the quality and browning of the product.

The composition according to the present invention is preferably frozen. Due to the freezing process, it is possible to inhibit the cholinesterase activity contaminating the composition, and choline esters can be conserved for a long time. The composition according to the present invention is preferably part or all of the fruit of a frozen Solanaceae Solanum eggplant species (Solanum melongena).

In one embodiment, the present invention relates to a method for producing a composition for oral ingestion having a blood pressure-lowering effect and/or an anti-stress effect, in which a choline ester is used as an active ingredient.

This method comprises a step for making an edible plant into a lyophilized powder and/or a hot-air dried powder or extract, and a step for dispensing the lyophilized powder and/or hot-air dried powder or extract so that the choline ester content is a predetermined amount.

This predetermined amount may be 5 μg to 250 mg, preferably 5 μg to 50 mg, and more preferably 10 μg to 50 mg. Moreover, in one embodiment, the predetermined amount may also be, for example, 5 to 500 μg, 5 to 250 μg, 10 to 50 μg or 25 μg.

The method according to the present invention may further comprise a step for heating the edible plant. Heating can be done in a microwave oven or by boiling in hot water. For example, when heating with a 550 W microwave oven, heating per 100 g of edible plants may be performed for 1 to 15 minutes, preferably 2 to 10 minutes, more preferably 4 to 6 minutes. Moreover, when boiling in hot water, it is preferred to heat in hot water of 90 to 100° C. By thus heating the edible plants, sterilization can be carried out and choline esters in edible plants can be increased.

While spoilage due to microorganisms is usually avoided by lyophilisation or by hot air drying, in one embodiment, the method according to the present invention is characterized in that edible plants are further heated (bactericidal activity and increase in choline esters).

The method according to the present invention may further comprise a step for suspending the lyophilized powder and/or hot air-dried powder of an edible plant in water, and a step for adding an acid to the suspension obtained. The suspension to which acid has been added is adjusted, for example, to a pH of 5.5 to 4.5, preferably of 5.4 to 4.6. By adjusting the pH in this manner, it is possible to stabilize the choline ester and to obtain a composition (suspension) with excellent long-term storability.

The present invention also relates to a method for producing an extract for oral ingestion having a blood pressure-lowering effect and/or anti-stress effect and comprising a choline ester as an active ingredient.

The method according to the present invention comprises a step for extracting an edible plant or a lyophilized powder and/or a hot air-dried powder of an edible plant with ethanol or hydrous ethanol.

More specifically, the method according to the present invention may comprise an extract in which choline esters are concentrated, which is obtained by adding ethanol or hydrous ethanol to a lyophilized powder and/or a hot air-dried powder of an edible plant, or by adding ethanol to a fresh edible plant and by grinding it to remove residues.

When extracting with hydrous ethanol, the ethanol concentration of the hydrous ethanol is not particularly limited, but it can be appropriately selected from the point of view of extraction rate or concentration ratio of choline esters, and the like. The ethanol concentration of hydrous ethanol may, for example, be 10% (w/w) or more, preferably 10 to 99% (w/w), more preferably 25 to 60% (w/w), or 95% (w/w) or more, particularly preferably 30 to 60% (w/w), or 99% (w/w) or more.

L-ascorbic acid may be added to the ethanol or aqueous ethanol used for extraction; L-ascorbic acid is added, for example, at a rate from 1 to 5 wt %, preferably 3 wt %.

The method according to the present invention may comprise a step for adjusting the choline ester content of the extract to 5 μg to 50 mg.

The present invention also relates to a composition for oral ingestion having a blood pressure-lowering effect and/or an anti-stress effect and comprising a choline ester as an active ingredient, which is prepared by the above method.

The dry powder relating to the composition according to the present invention is preferably a powder passed through a sieve with an appropriate mesh size. The composition according to the present invention preferably comprises, for example, a lyophilized powder and/or a hot air-dried powder capable of passing through a 20 mesh sieve.

Here, the hot air-dried powder can be prepared, for example, by drying an edible plant by exposing it to hot air of about 90° C. for about 1 to 2 hours, and by grinding it to a powder.

The composition according to the present invention can be used as an active ingredient of various functional health foods or pharmaceutical compositions.

In the case of food, it can be used as a food composition in combination with appropriate food additives. Moreover, the present invention is not limited to such a food composition, it can also be provided in embodiments in which it is orally ingested on a daily basis, for example, as a beverage blended with green tea, black tea, Oolong tea, cereal tea, and the like, or as a food blended in biscuits, bread, candy, and the like. Furthermore, the composition according to the present invention can also be used as a so-called food supplement in an appropriate dosage form according to the formulation of the following pharmaceuticals.

When the composition according to the present invention is made into a pharmaceutical product, it can be combined with appropriate pharmaceutical additives and can be used in various dosage forms according to general procedures of preparation for pharmaceuticals. Examples of such dosage forms include oral administration preparations including, for example, solid preparations such as powders, granules, capsules, pills and tablets, or the like, and liquid preparations such as solutions, suspensions and emulsions, or the like.

When the composition according to the present invention is used as a food product, examples of usage include not only general foods and beverages, but also functional health foods to promote health by exhibiting specific functions.

Examples of specific embodiments in such cases include supplements such as capsules, tablets, powders, and granules, and the like, bakery foods such as breads, cakes and cookies, and the like, seasonings such as sauces, soups, dressings, mayonnaise, and the like, dairy products such as milk, yogurt, cream, and the like, confectioneries such as chocolate, candies, and the like, or a variety of beverages such as green tea, black tea, Oolong tea, barley tea, cereal tea, juice, vegetables drinks, milk drinks, soft drinks and carbonated beverages, and the like, all containing the composition of the present invention as an active ingredient.

When the composition according to the present invention is used as an active ingredient of a pharmaceutical composition, its dose differs depending on the ratio of each component, and on various factors such as a patient's age, weight, sex, symptoms, administration method, and the like, but in the case of daily doses for oral administration of an adult, generally, the choline ester content can be selected in the range of 5 μg to 50 mg, and in one embodiment, in the range of 5 μg to 500 μg. The dose can be increased or decreased as appropriate depending on the degree of improvement of the symptoms. Regarding the frequency of administration, it can be administered from one to several times a day.

When the composition according to the present invention is used as a food product, its intake amount can be selected in accordance with the above case of pharmaceuticals used for oral administration. However, opposed to pharmaceuticals, in the case of food and beverages, the amount and frequency of the doses are not particularly limited, therefore, as long as there are no particularly severe symptoms, the intake amount may be selected without the limitation of the above range by taking into consideration the aim of maintaining health, as well as taste and palatability.

### EXAMPLES

Hereinafter, embodiments of the present invention will be described by way of examples and test examples; however, the present invention is not limited to the following examples. Moreover, the meanings of abbreviations in the examples are as follows. Furthermore, the generic names of EN, AcCh, BuCh, Ch, LaCh and PrCh are choline compounds. EtOH: ethanol. EN: (2-aminoethyl) trimethylammonium pivaloylamide, AcCh: acetylcholine, BuCh: butyrylcholine, Ch: choline, LaCh: lactoylcholine, PrCh: propionylcholine.

### [Test Materials and Methods]

### 1. Analysis Samples

As analysis samples the agricultural products according to Table 1 were obtained.

TABLE 1
Cultivar Name and Cultivation Site/Vendor of the Analysis Samples

| Crop Name | Cultivar/Species Name | Cultivation site/Vendor |
|---|---|---|
| Cucumber | ‘Zubari 163’ | Tokoo, Oaza Soga, Shiojiri-shi, Nagano-ken |
| Tomato | ‘Rinka 409’ | Tokoo, Oaza Soga, Shiojiri-shi, Nagano-ken |
| Paprika | ‘Special’ | Tokoo, Oaza Soga, Shiojiri-shi, Nagano-ken |
| Bell Pepper | Bell-masari | Tokoo, Oaza Soga, Shiojiri-shi, Nagano-ken |
| Asparagus | ‘Welcome’ | Tokoo, Oaza Soga, Shiojiri-shi, Nagano-ken |
| Mountain | Nagaimo | Tokoo, Oaza Soga, Shiojiri-shi, Nagano-ken |
| Yam | | |
| Cabbage | ‘Shinshu 868’ | Tokoo, Oaza Soga, Shiojiri-shi, Nagano-ken |
| Lettuce | ‘Shinano Hope’ | Tokoo, Oaza Soga, Shiojiri-shi, Nagano-ken |
| Carrot | ‘Koyo No. 2’ | Tokoo, Oaza Soga, Shiojiri-shi, Nagano-ken |
| Shishito | ‘Manganji Togarashi’ | Nishi Minowa, Ina-shi, |
| pepper | ‘Shinano Dolce’ | Nagano-ken |
| Apple | | Azusakawa, Matsumoto-shik, |
| | | Nagano-ken |
| Nashi pear | ‘Nijisseiki’ | Misuzu, Ina-shi, Nagano-ken |
| Grape | ‘Nagano Purple’ | Minami Minowa, Nagano-ken |
| Sprouts | Daikon sprout | Salad Cosmo Inc. |
| | Broccoli sprout | Salad Cosmo Inc. |
| | Alfalfa bean sprout | Salad Cosmo Inc. |
| | Soybean sprout | Salad Cosmo Inc. |
| | Buckwheat sprout | Salad Cosmo Inc. |
| Bamboo | Moso Bamboo | Toyooka-mura, Shiminnaina- |
| shoot | | gun, Nagano-ken |
| Eggplants | ‘Sen-ryo Nigo’ | Tokoo, Oaza Soga, Shiojiri-shi, Nagano-ken |
| | ‘Senshu’ water | Hannan-shi, Osaka-fu |
| | eggplant | |
| | ‘Batten’ eggplant | Uki-shi, Kumamoto-ken |
| | ‘Koryo’ salad eggplant | Koryo-cho, Kitakatsuragi- |
| | | gun, Nara-ken |
| | ‘Higomurasaki’ | Takamori, Aso-gun, |
| | | Kumamoto-ken |
| | ‘Oonaga’ eggplant | Kamoto, Yamaga-shi, |
| | | Kumamoto-ken |
| | ‘Chikuyo’ | Kumamoto-shi, Kumamoto- |
| | | ken |

### 2. Extraction Method

### (1) Preparation of Samples

Immediately after obtaining the fresh agricultural products (analysis samples), the surface was washed with tap water. After wiping off the moisture, only the edible portion was sliced with a kitchen knife to a width of 1 to 3 cm as necessary. The edible parts were lyophilized with a freeze dryer (FDU-2000, Tokyo Rikakikai Co., Ltd.). The lyophilizate was ground in a mill mixer (MASTER, Tokyo Unicom Co., Ltd.) and made into powder.

### (2) Preparation of Reagent

Sodium dihydrogenphosphate (59.99 mg) and disodium hydrogenphosphate (70.98 mg) were weighed and dissolved in pure water (100 mL) to prepare a 10 mM phosphate buffer solution.

EN (0.80 mg) was dissolved in the 10 mM phosphate buffer solution (1 mL) to prepare a 800.00 μg/mL solution and then diluted 100 times to prepare a 8.00 μg/mL solution, which was used as an EN internal standard.

### (3) Shaking Extraction

The lyophilizate (10 mg) was weighed into a 2 mL tube and the EN internal standard (10 μL) was added. The 10 mM phosphate buffer solution (190 μL) was added and the mixture was stirred for 3 minutes with a vortex (FLX-S, FRONT LAB, As One Corporation), and then centrifuged (1000×g, at ambient temperature, for 3 minutes) with a centrifuge (CFM-200, Iwaki Co., Ltd.) to obtain a supernatant. The 10 mM phosphate buffer solution (200 μL) was again added to the residue, and the operation of stirring, centrifugation and collection of supernatant was repeated twice. All of the collected supernatants were combined (about 600 μL) to prepare an extraction sample.

### (4) Solid Phase Extraction

A weakly acidic cation exchange cartridge Inert Sep CBA 100 mg/1 mL (GL Sciences Co., Ltd.) was used as solid phase extraction cartridge. The solid phase extraction cartridge activated with methanol (1 mL) and pure water (1 mL) was equilibrated with the 10 mM phosphate buffer (8 mL), and the extraction sample (about 600 μL), prepared under (3), above was added. It was stabilized with 10 mM phosphate buffer solution (600 μL), washed with pure water (2.5 mL), and eluted with hydrochloric acid (500 μL).

### (5) Quantitative Sample Preparation

The eluate (500 μL) eluted with hydrochloric acid by solid phase extraction was accurately filled up to 1 mL with LC-MS/MS analysis solvent using 1 mL volumetric flasks and divided into 3 parts of 300 μL. A mixed solution of choline compounds was added to each flask, and an LC-MS/MS analysis solvent was added (Table 2) so that the eluate was diluted 2-fold (Table 2) to prepare quantitative samples.

TABLE 2
Amount of each Solution added to the Quantitative Sample

| | Added Amount (μL) for each | | |
|---|---|---|---|
| | Standard Sample | | |
| Added Solution | A | B | C |
| Eluate | 300 | 300 | 300 |
| Mixed Solution of | 0 | 51 | 102 |
| Choline Compounds | | | |
| LC-MS/MS Analysis | 300 | 249 | 198 |
| Solvent | | | |
| Total Liquid Volume | 600 | 600 | 600 |

The mixed solution of choline compounds was prepared as shown in Table 3, and the added concentration of each stock solution of choline compound was determined based on the analysis result of the sample without a mixed solution of choline compounds (Table 2-A). The choline compound stock solution was diluted with LC-MS/MS analysis solvent and adjusted to the respective concentrations. If there were undetected choline compounds, only a volume of LC-MS/MS analysis solvent equivalent to that not comprising those compounds was added.

TABLE 3
Method for preparing a Choline Compound Mixed Solution

| Choline Ester Compound | Mixed Solution |
|---|---|
| Solution | Volume (μL) |
| EN Stock Solution | 40 |
| AcCh Stock Solution | 10 |
| BuCh Stock Solution | 20 |
| Ch Stock Solution | 10 |
| LaCh Stock Solution | 70 |
| PrCh Stock Solution | 20 |
| Total Solution Volume | 170 |

### 3. LC-MS/MS Analysis

### (1) LC-MS/MS Analysis Conditions

A YMC-Triart PFP (4.6 mm×250 mm, 5 μm, YMC Co., Ltd.) was used as column. LC-MS/MS analysis was carried out by using water containing 0.01% formic acid-33% methanol as an analysis solvent under the conditions of a flow rate of 0.5 mL/min (LC), 0.3 mL/min (MS), an injection volume of 50 μL, a separation temperature of 40° C., an analysis time of 30 min, an ionization mode of ESI+⋅MRM, a Capillary Voltage of 3500 V, a Cone Voltage of 10 V, a Collision Voltage of 10 V, an N₂ gas flow (desolvation) of 600 L/hr, an N₂ gas flow (cone) of 50 L/hr, an N₂ source temp of 120° C., and an N₂ desolvation temp of 350° C., while using an ACQUITY UPLC [UPLC, Waters Corp.]—Quattro micro API [MS, Waters Corp.]. The MRM mode designation m/z of each choline compound is shown in Table 4.

TABLE 4
MRM Mode setting Condition

| Choline Compound | Parent (m/z) | Daughter (m/z) |
|---|---|---|
| EN | 187.18 | 128.15 |
| AcCh | 146.10 | 87.00 |
| BuCh | 174.10 | 115.00 |
| Ch | 104.20 | 60.20 |
| LaCh | 176.10 | 117.05 |
| PrCh | 160.10 | 101.00 |

### (2) Standard Addition Method

A calibration curve was prepared from the peak area values of the chromatograph obtained by LC-MS/MS analysis, and the choline compounds were quantified by a standard addition method. The concentration of each choline compound was corrected with the recovery rate calculated from the calibration curve of the EN internal standard to calculate the accurate choline compound concentration in the quantitative samples. The amount of each choline compound (μg/100 g FW) was calculated per 100 g fresh weight from the yield before and after lyophilization after converting the obtained concentration into the content (mg/g DW) in the lyophilizate.

### [Experiment 1] Fresh Weight and Dry Weight

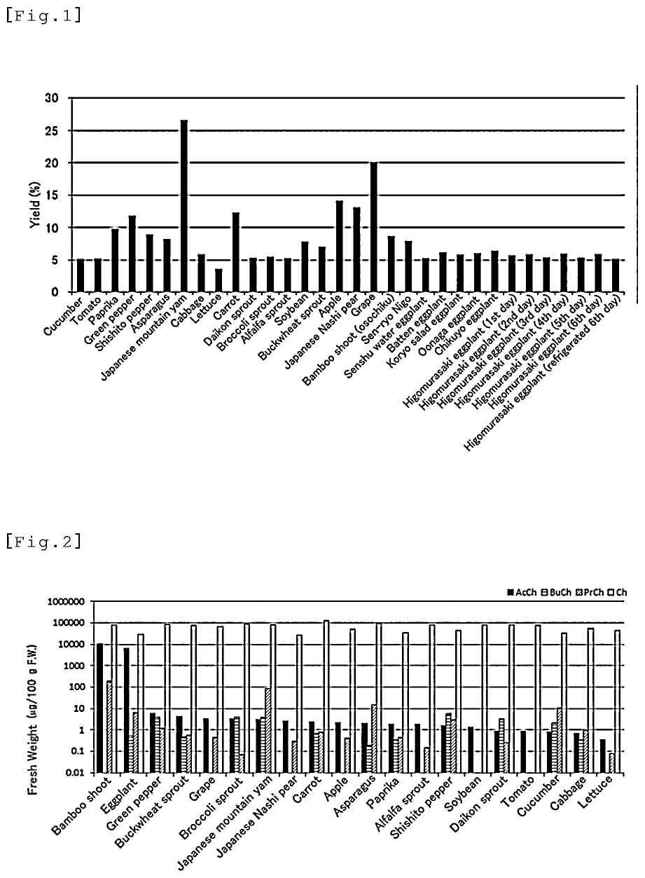

The fresh weight and the dry weight of various fresh agricultural products was obtained and the yields before and after lyophilization were calculated. The results are shown in FIG. 1.

As shown in FIG. 1, the yield of lettuce (Shinano Hope) (fresh weight: 79.19 g, dry weight: 2.85 g) was the lowest at 3.60%, and the yield of Japanese mountain yams (Nagaimo) (fresh weight: 79.76 g, dry weight: 21.23 g) was the highest at 26.62%. The yield for most agricultural products was about 5 to about 10%.

### [Experiment 2] Quantification of Choline Compounds in Each Fresh Agricultural Product

By the above method, choline compounds were extracted from 20 types of fresh agricultural products and analysis of the five types of choline compounds AcCh, Ch, BuCh, PrCh, LaCh was performed by LC-MS/MS with n=3. The results are shown in FIG. 2.

AcCh and Ch were detected in all of the tested agricultural products; while, LaCh was not detected in all of the tested agricultural products. Moreover, BuCh was detected in agricultural products other than tomatoes, lettuce, alfalfa sprouts, bean sprouts, buckwheat sprouts, apples, Japanese Nashi pears, grapes, and bamboo shoots. PrCh was detected in agricultural products other than tomatoes and bean sprouts.

As a result of the quantification in this experiment, the AcCh detected in bamboo shoots and eggplants was more than 1000 times higher than in 18 other types of fresh agricultural products. The AcCh content was highest in bamboo shoots.

### [Experiment 3] Quantification of Choline Compounds in 6 Varieties of Eggplants

In Experiment 2, the difference in choline compound content between cultivars was investigated in eggplants, which are rich in AcCh, can be eaten as fresh produce, and can be grown annually. By the above method, choline compounds were extracted from 6 varieties of eggplants, and analysis of the 5 types of choline compounds AcCh, Ch, BuCh, PrCh, LaCh was performed by LC-MS/MS with n=3. The results are shown in Table 5. LaCh was not detected in any cultivar. Moreover, among the six varieties, Higomurasaki contained the most AcCh.

TABLE 5
Quantification Value of each Choline Compound in 6 Varieties of Eggplants

| Variety | AcCh | BuCh | Ch | PrCh |
|---|---|---|---|---|
| Higomurasaki | 5.53 × 10³ ± 171 | 2.85 ± 0.09 | 3.09 × 10⁴ ± 720 | 7.78 ± 0.18 |
| Chikuyo | 5.47 × 10³ ± 88 | 1.43 ± 0.04 | 3.38 × 10⁴ ± 1419 | 6.97 ± 0.15 |
| Senshu water eggplant | 5.15 × 10³ ± 102 | 7.40 × 10¹ ± 0.04 | 2.18 × 10⁴ ± 743 | 5.65 ± 0.04 |
| Koryo salad eggplant | 4.24 × 10³ ± 107 | 1.28 ± 0.05 | 1.96 × 10⁴ ± 338 | 3.71 ± 0.07 |
| Batten eggplant | 3.06 × 10³ ± 50 | 6.04 × 10⁻¹ ± 0.08 | 3.63 × 10⁴ ± 1748 | 1.53 ± 0.01 |
| Oonaga eggplant | 2.79 × 10³ ± 112 | 2.94 ± 0.04 | 3.56 × 10⁴ ± 848 | 5.90 ± 0.10 |

(μg/100 g F.W)

While the AcCh in eggplants has up to now been quantified by high performance liquid chromatography electrochemical detection (HPLC-ECD) method, and a content of 6.09×10³ μg/100 g FW has been reported (Non-Patent Document 7), the quantification result by LC-MS/MS analysis in this experiment has also arrived at the same AcCh content.

[Experiment 4] The Change over Time of the Choline Compound Content in Eggplants

The storage period of eggplant fruits is generally 2 to 3 days at ambient temperature and about one week at a temperature of 7 to 10° C.; however, storage at 6° C. or less is not recommended because chilling injuries such as pitting, in which the skin collapses into craters, browning and black depressions, and the like, may occur. The suitable storage temperature of eggplant fruits is 10° C. or more to 20° C. or less.

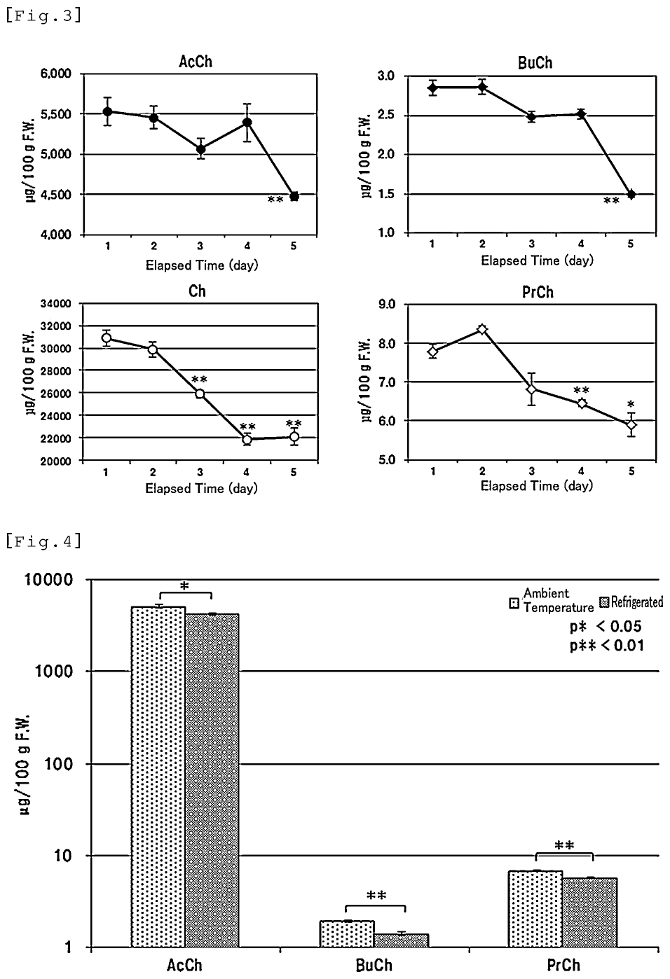

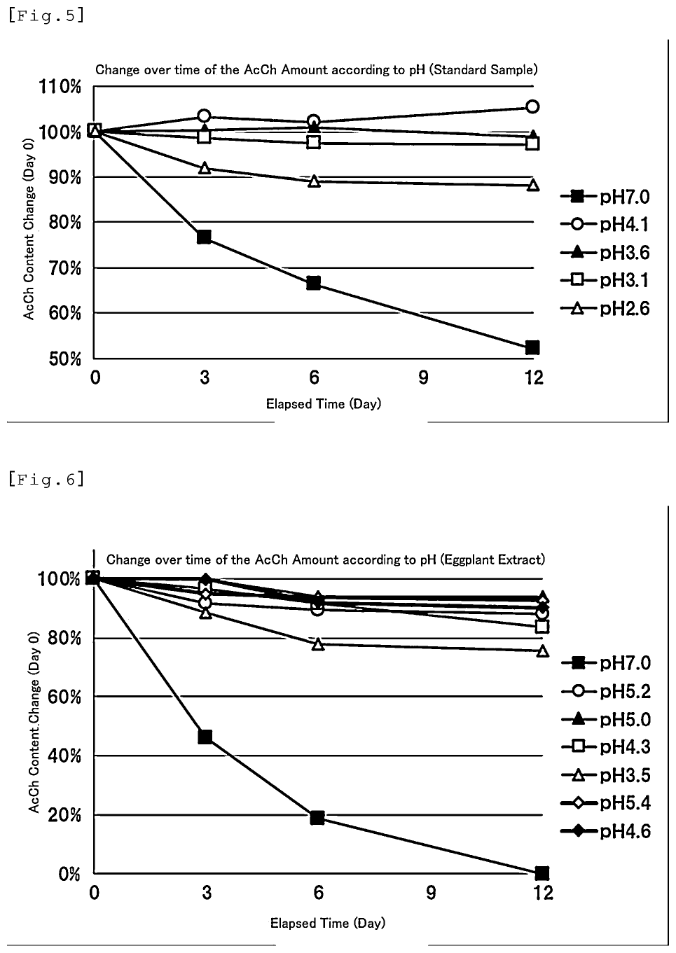

In this experiment, to investigate the change over time in the amount of choline compounds in the case of the ‘Higomurasaki’ eggplant cultivars stored at ambient temperature, the eggplant fruits were wrapped in newspaper and stored in a dark place at ambient temperature, about 10 g was sampled once each day from the same fruit. A separate fruit was divided into two parts; one part was sampled after 6 days of storage at ambient temperature. Moreover, the other half of the sample divided into two parts was prepared by wrapping it into newspaper and putting it in refrigerated storage for 6 days, and by the above method, choline compounds were extracted, and analysis of the 5 types of choline compounds AcCh, Ch, BuCh, PrCh, LaCh was performed by LC-MS/MS with n=3. The results are shown in Table 6 and in FIG. 3 and FIG. 4.

TABLE 6
Quantification Values of each Choline Compound during Storage at Ambient
Temperature and Refrigerated Storage of ‘Higomurasaki’ Eggplants

| Days of Storage | AcCh | BuCh | Ch | PrCh |
|---|---|---|---|---|
| 1 at Ambient Temperature | 5.53 × 10³ ± 171 | 2.85 ± 0.09 | 3.09 × 10⁴ ± 720 | 7.78 ± 0.18 |
| 2 at Ambient Temperature | 5.45 × 10³ ± 145 | 2.86 ± 0.10 | 2.99 × 10⁴ ± 645 | 8.35 ± 0.09 |
| 3 at Ambient Temperature | 5.07 × 10³ ± 122 | 2.48 ± 0.06 | 2.59 × 10⁴ ± 322 | 6.82 ± 0.41 |
| 4 at Ambient Temperature | 5.39 × 10³ ± 239 | 2.52 ± 0.07 | 2.19 × 10⁴ ± 527 | 6.45 ± 0.08 |
| 5 at Ambient Temperature | 4.48 × 10³ ± 51 | 1.49 ± 0.04 | 2.21 × 10⁴ ± 785 | 5.90 ± 0.30 |
| 6 at Ambient Temperature | 5.15 × 10³ ± 223 | 1.97 ± 0.06 | 2.54 × 10⁴ ± 966 | 6.97 ± 0.10 |
| 6 in Refrigerated Storage | 4.26 × 10³ ± 113 | 1.44 ± 0.07 | 2.16 × 10⁴ ± 837 | 5.81 ± 0.10 |

(μg/100 g F.W)

LaCh was not detected in any of the samples. The content of each choline compound tended to gradually decrease with the passage of storage days (FIG. 3). Moreover, in this experiment, chilling injuries such as pitting did not occur in the sample in refrigerated storage, but the residual amount of choline esters was higher in the sample stored at room temperature than in the sample in refrigerated storage (FIG. 4).

From the results of this experiment it can be seen that there is the trend that the choline ester content gradually declines with the passage of days when eggplants are stored for 5 days at ambient temperature. However, since 80% or more of AcCh on the first day remained in the sample stored at room temperature after 5 days, it can be considered that there was no drastic change in the amount of AcCh in the eggplant stored at room temperature, and that the stability was high.

### [Experiment 5] AcCh Stability Under Acidic Conditions

A method for stabilizing choline esters in eggplants was examined. Generally, ester compounds are easily hydrolyzed under basic conditions and relatively stable under acidic conditions. Furthermore, 99% or more of the choline ester contained in eggplant is AcCh. Therefore, firstly, the concentration change over time of an AcCh standard sample was examined under neutral and acidic conditions. The neutral solution (pH 7.0) was a phosphate buffer solution, and the acidic solutions (pH 4.1, pH 3.6, pH 3.1, pH 2.6) were prepared by adding acetic acid; the pH solutions with an AcCh concentration of 1.9 mg/mL were allowed to remain at ambient temperature (23 to 25° C.) for 0, 3, 6, 12 days, and then the AcCh content was quantified. The results are shown in Table 7 and FIG. 5.

TABLE 7
Change over Time of the AcCh Amount in
Aqueous pH Solutions (Standard Samples)

| Elapsed | | | | | |
|---|---|---|---|---|---|
| Days | pH 7.0 | pH 4.1 | pH 3.6 | pH 3.1 | pH 2.6 |
| 0 | 100% | 100% | 100% | 100% | 100% |
| 3 | 76.6% | 103.3% | 100.3% | 98.6% | 91.8% |
| 6 | 66.5% | 101.9% | 101.0% | 97.3% | 89.1% |
| 12 | 52.3% | 105.3% | 98.7% | 97.0% | 88.0% |
| | Phosphate | 0.0015% | 0.015% | 0.15% | 1.50% |

| | |
|---|---|
| Buffer | Acetic Acid Concentration |

The pH 7.0 is a phosphate buffer solution and the acidic solutions were prepared by adding acetic acid.

Under neutral conditions, AcCh decomposed over time and decreased to about half after 12 days. On the other hand, at pH 4.1 (acetic acid concentration 0.0015%), the AcCh content was stable, and there was the tendency for AcCh to increase after 12 days had passed. It has been made clear that at pH 4.1 to pH 3.1, AcCh remained stable at 97% or more even after 12 days.

At pH 2.6, the stability of AcCh decreased somewhat and the residual rate after 12 days was 88%.

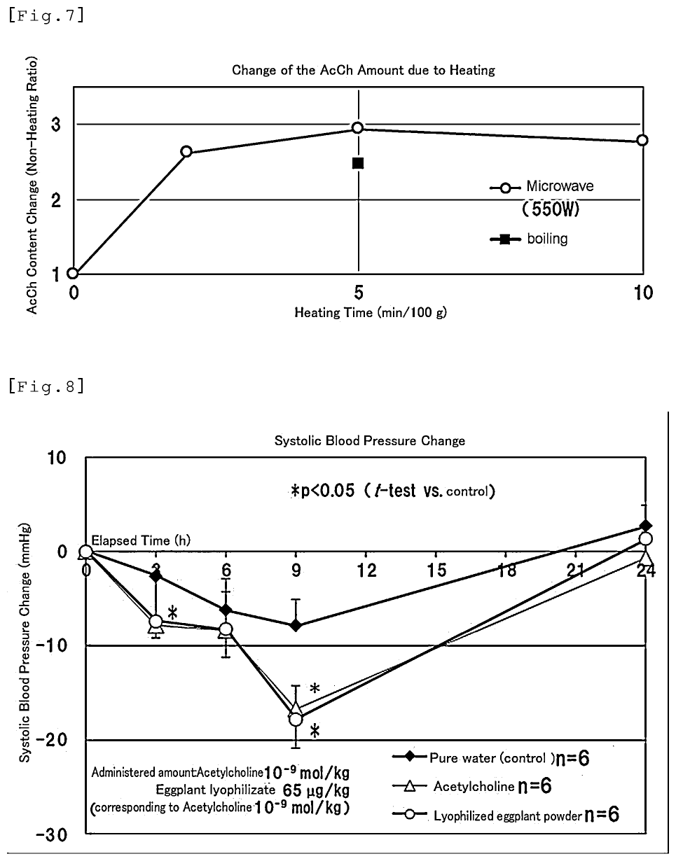

Next, the stability of AcCh contained in eggplant extract was examined under neutral and acidic conditions. The neutral solution (pH 7.0) was a phosphate buffer solution, and the acid solutions (pH 5.2, pH 5.0, pH 4.3, pH 3.5) were prepared by adding acetic acid, while the acidic solutions (pH 5.4, pH 4.6) were prepared by adding ascorbic acid: the pH solutions, to which an eggplant extract was added so that the AcCh concentration became 0.040 mg/mL, were allowed to remain at ambient temperature (23 to 25° C.) for 0, 3, 6, 12 days, and then the AcCh content was quantified. The results are shown in Table 8 and FIG. 6.

TABLE 8
Change over Time of the AcCh Amount in Aqueous pH Solutions (Eggplant Extracts)

| Elapsed | | | | | | | |
|---|---|---|---|---|---|---|---|
| Days | pH 7.0 | pH 5.2 | pH 5.0 | pH 4.3 | pH 3.5 | pH 5.4 | pH 4.6 |
| 0 | 100% | 100% | 100% | 100% | 100% | 100% | 100% |
| 3 | 45.8% | 91.7% | 99.7% | 96.7% | 88.4% | 94.9% | 99.8% |
| 6 | 18.5% | 89.6% | 94.1% | 91.6% | 77.8% | 93.6% | 92.0% |
| 12 | 0% | 88.1% | 93.8% | 83.5% | 75.4% | 92.8% | 90.2% |
| | Phosphate | 0.0015% | 0.015% | 0.15% | 1.50% | 0.0015% | 0.015% |

| | | |
|---|---|---|
| Buffer | Acetic Acid | Ascorbic Acid |
| | Concentration | Concentration |

The pH 7.0 is a phosphate buffer solution and the acidic solutions were prepared by adding acetic acid or ascorbic acid.

Under neutral conditions, AcCh in eggplant extract was unstable; more than half of it had decomposed after 3 days, and nearly all AcCh had decomposed after 12 days. Under acidic conditions, AcCh was relatively stable; at pH 5.4 to 4.6, 88% or more of AcCh remained even after 12 days, but in the solution adjusted with acetic acid to pH 3.5, 24.6% of AcCh had decomposed after 12 days. AcCh in eggplant was stable at room temperature under acidic conditions of pH 5.4 to 4.6.

From the above results it is clear that it is possible to stabilize choline ester in agricultural products such as eggplants by adjusting the pH from 5.5 to 4.5 by adding an organic acid, or the like. As long the pH is within this range when a suspension is prepared, the acid may be added at an appropriate time.

### [Experiment 6] Change in AcCh Content in Eggplants Due to Heating

In order to investigate how the AcCh content in eggplants is affected by heating, ‘Ryoma’, a typical long ovular eggplant variety, was cut to a thickness of 2 to 3 cm, wrapped in a cloth, heated in a microwave oven (550 W) while varying the heating time, allowed to cool at ambient temperature to quantify the AcCh content in the lyophilized samples. Moreover, the AcCh content in eggplants that were boiled for 5 minutes in boiling water were also examined. The temperature was measured and recorded immediately after the heat treatment. The results are shown in Table 9 and FIG. 7.

TABLE 9
Change in AcCh Content in Eggplants due to Heating

| | Microwave Oven (550 W) | | Boiling | |
|---|---|---|---|---|
| | AcCh | | AcCh | |
| Heating Time | Increase | Temperature | Increase | Temperature |
| (min/100 g) | Rate | (° C.) | Rate | (° C.) |
| 0 | 100% | 25.0 | | 25.0 |
| 2 | 262% | 91.1 | | |
| 5 | 294% | 92.6 | 248% | 95.6 |
| 10 | 277% | 87.8 | | |

It has become clear that choline ester in eggplant increases when heated in a microwave oven. When heating for 5 minutes per 100 g, AcCh increased most and, compared to the non-heated sample, the AcCh content increased 2.9 times. Compared to the non-heated sample, the AcCh content increased 2.6 times when heated for 2 minutes per 100 g, and 2.8 times when heated for 10 minutes per 100 g. The internal temperature reached 91.1° C. when heating for 2 minutes and 92.6° C. when heating for 5 minutes, and decreased to 87.8° C. during 10 minutes by water evaporation. When boiled for 5 minutes per 100 g, the AcCh content increased 2.5 times, and the internal temperature reached 95.6° C.

From the above results it can be inferred that the AcCh content generally increases during the heating process and is kept stable to some extent. Even if the heating conditions are varied, the AcCh content can be expected to increase if the temperature change is similar. Moreover, sterilization can be performed in this step.

From the results of Experiments 4 to 6, it has become clear that acetylcholinesterase (EC number 3.1.1.7), which is a choline ester-degrading enzyme, influences the choline ester content. As described in Non-Patent Document 3, acetylcholinesterase is widely present in plants. It is considered that the AcCh decrease shown in Table 8 is mainly caused by acetylcholine-esterase degradation. It is further thought that AcCh degradation was inhibited at a low pH at which the enzyme activity declines, and that more AcCh degraded at neutral conditions close to the optimum pH of acetylcholine-esterase of 8.0 to 8.5. Furthermore, when heated, acetylcholine-esterase is inactivated; therefore it is considered that AcCh was not degraded, and that it was concentrated when moisture evaporated. In other words, processing technology for controlling acetylcholine-esterase is important for eggplants and processed eggplant products. Therefore, the change in AcCh content of frozen eggplants and processed eggplant products in which eggplant acetylcholine-esterase does not function was investigated.

### [Experiment 7] Change in AcCh Content in Frozen Fresh Eggplants and Frozen Heated Eggplant Experiment

Fresh eggplants (cut to about 10 g) and heated eggplants (cut to about 10 g and heated for 5 minutes per 100 g in a 550 W microwave oven) were frozen at −20° C., samples were taken out every month and frozen and lyophilized; then the AcCh content was quantified and changes in AcCh content were examined during 6 months.

The fresh eggplant sample and the dried eggplant sample were each cut from the same eggplant fruit. The results are shown in Table 10.

TABLE 10
Change in Acetylcholine Content in Eggplants due to Freezing

| | Freezing Period (Month) | | | | | | |
|---|---|---|---|---|---|---|---|
| | Before | | | | | | |
| | Freezing | 1 | 2 | 3 | 4 | 5 | 6 |
| Fresh Eggplants | 100% | 99.2% | 100.3% | 101.7% | 99.0% | 99.3% | 98.3% |
| Heated Eggplants | 100% | 101.2% | 102.0% | 101.0% | 103.1% | 100.8% | 101.9% |

It has become clear that AcCh in fresh eggplants and heated eggplants hardly changed due to freezing and that it was possible to maintain choline ester in eggplants at a stable level for a long period of time by freezing. The result was that, in fresh eggplants, the activity of choline-esterase, which is a cause for degrading choline ester in eggplants, can be inhibited by freezing. It is considered that the inhibition of choline-esterase activity by freezing was sufficient because the AcCh content change was almost equivalent to the result of the heated eggplants in which cholinesterase was inactivated by heating in advance.

### [Experiment 8] Preparation of Eggplant Extract

Eggplant extract was prepared by using lyophilized eggplant powder or fresh eggplants. The method for preparing extract with a lyophilized eggplant powder is as follows.

Lyophilized eggplant powder (1 g) was weighed in a centrifugal tube (50 mL capacity), a 10-fold weight (10 g) of a solvent was added in which the EtOH concentration was varied by 10% each from 10% to 100%, and stirred by shaking at 3000 rpm at ambient temperature for 30 minutes, after which the supernatant was obtained by filtration. The supernatant was transferred to an eggplant flask (200 mL), then pure water (10 g) was added and the mixture was concentrated using an evaporator. When the liquid amount was reduced to about ⅕, the content was completely transferred to a centrifuge tube (15 mL) and lyophilized. The lyophilizate was used as eggplant extract and the yield was determined and quantified by the methods described in “2. Extraction Method” and “3. LC-MS/MS Analysis”. In the extractions using 50% EtOH, L-ascorbic acid was added to prepare the extract. The results are shown in Table 11.

TABLE 11
Results of the Eggplant Extract Preparation Experiment (Lyophilized Eggplant Powder Material)

| EtOH Concentraton (%) | 10 | 20 | 30 | 40 | 50 | 60 | 70 | 80 | 90 | 100 | 50** | 100** |
|---|---|---|---|---|---|---|---|---|---|---|---|---|
| Extraction yield (%)* | 53.7 | 46.1 | 46.4 | 45.9 | 54.3 | 57.7 | 45.1 | 42.8 | 34.8 | 15.6 | 61.9 | 18.8 |
| Choline Ester | 1.6 | 1.9 | 2.2 | 2.2 | 1.9 | 1.6 | 1.8 | 1.9 | 2.4 | 2.9 | 2.4 | 3.6 |
| Concentration (mg/g) | | | | | | | | | | | | |
| Choline Ester | 61.7 | 60.6 | 71.9 | 70.4 | 73.6 | 65.3 | 55.9 | 57.2 | 60.0 | 32.3 | 105.2 | 47.8 |
| Extraction Rate (%) | | | | | | | | | | | | |
| Concentration | 1.2 | 1.3 | 1.6 | 1.5 | 1.4 | 1.1 | 1.2 | 1.3 | 1.7 | 2.1 | 1.7 | 2.6 |
| Ratio (Times) | | | | | | | | | | | | |
| Colour | Dark | Dark | Dark | Dark | Dark | Dark | Dark | Dark | Dark | Dark | Deep | Deep |
| | Brown | Brown | Brown | Brown | Brown | Brown | Brown | Brown | Brown | Brown | Purple | Purple |

*Lyophilized eggplant powder standard,
**Addition of 3% of ascorbic acid

When fresh eggplants were used, EtOH of each concentration was added to the cut fresh eggplants (10 g), the mixture was ground with a mill mixer, and prepared by the same method as the lyophilized eggplant powder above; the yield and the choline ester content were also determined by the same method. The added weight of EtOH was 0.5, 1 and 2 times the weight of the fresh eggplant. Moreover, an extract using the same amount of EtOH was prepared by adding L-ascorbic acid. The results are shown in Table 12.

TABLE 12
Results of the Eggplant Extract Preparation
Experiment (Fresh Eggplant Material)

| Added EtOH Weight (g) | 5 | 10 | 20 | 10** |
|---|---|---|---|---|
| Extraction Yield (%)* | 36.3 | 53.4 | 63.5 | 58.9 |
| Choline Ester | 1.1 | 2.0 | 2.6 | 2.2 |
| Concentration (mg/g) | | | | |
| Choline Ester | 28.3 | 75.6 | 116.8 | 91.7 |
| Extraction Rate (%) | | | | |
| Concentration | 0.78 | 1.4 | 1.9 | 1.6 |
| Ratio (Times) | | | | |
| Colour | Dark | Dark | Dark | Deep |
| | Brown | Brown | Brown | Purple |

*Fresh eggplant standard,
**Addition of 3% of ascorbic acid

An eggplant extract is a water-soluble semi-solid material in which the choline esters are more concentrated than in a lyophilized powder and/or a hot air-dried powder. When L-ascorbic acid is added to acidify a solution and inhibit oxidation, it is possible to stabilize the choline esters and prevent color changes in the extraction process.

### [Experiment 9] Single Oral Administration Test Using an Eggplant Lyophilizate in Spontaneously Hypertensive Rats

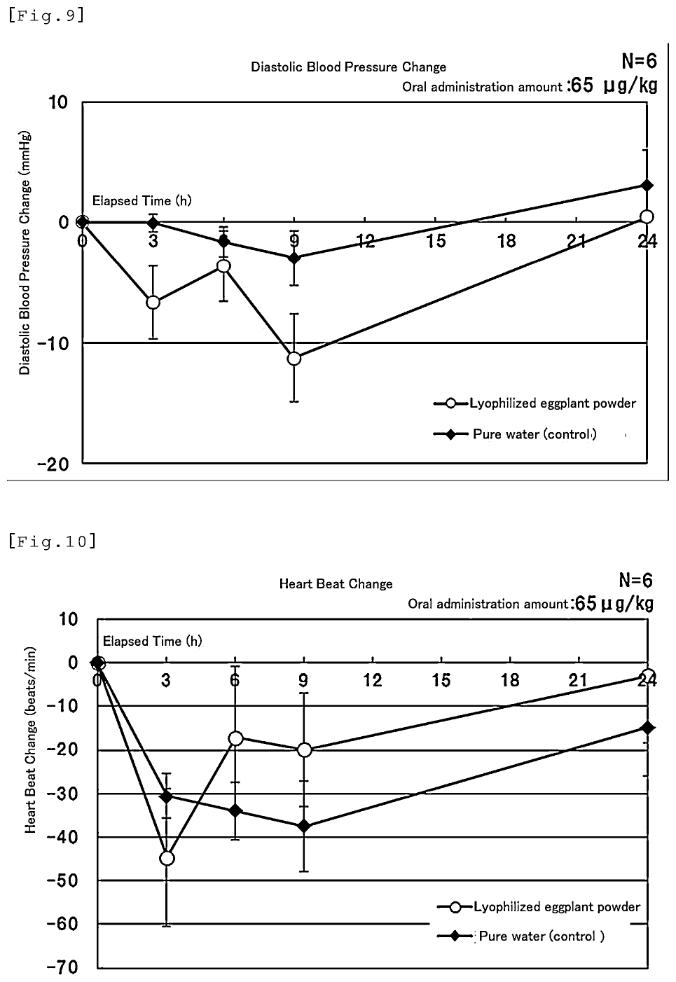

An eggplant lyophilizate was fed to spontaneously hypertensive rats (SHR) by single oral administration and the blood pressure-lowering effect was examined. After 14-week-old male SHRs (body weight 324 to 368 g) were conditioned for 1 week, they fasted for 12 hours, after which lyophilized eggplant powder suspended in water was fed by single oral administration using a tube. The lyophilized eggplant powder was prepared by washing a ‘Senshu’ water eggplant from Hannan-shi, Osaka-fu with water, by sterilizing the eggplant with hypochlorous acid, and by finely grinding the edible part after lyophilisation. More than 99% of the choline esters in the lyophilized eggplant powder were AcCh and the AcCh content was 2.25 mg/g DW. A dosage of 0.065 mg/kg of lyophilized eggplant powder corresponding to 1.00×10⁻⁹ mol (0.146 μg)/kg of AcCh was fed to 6 SHRs by single oral administration. In order to compare the blood pressure-lowering effect, an administration test of an AcCh standard sample of 1.00×10⁻⁹ mol/kg was performed in the same manner and the systolic blood pressure was measured. To a control group only water was administered under the same conditions. 0, 3, 6, 9, and 24 hours after the oral administration, systolic blood pressure, diastolic blood pressure and heart rate were measured by the tail cuff method using the non-invasive blood pressure measuring instrument Softron BP-98A (Softron Co., Ltd., Tokyo). The results of the single oral administration test of the lyophilized eggplant powder are shown in FIGS. 8 to 10.

Compared to the control group, a significant systolic blood pressure-lowering effect (p<0.05) occurred 3 to 9 hours after administration in the group to which the lyophilized eggplant powder was administered. The maximum blood pressure reduction of −17.8 mmHg occurred 9 hours after administration, which was −10.0 mmHg lower than in the control group. The acetylcholine 1.00×10⁻⁹ mol/kg administration showed almost the same blood pressure-lowering effect. Furthermore, the diastolic blood pressure showed a decreasing tendency, and the maximum blood pressure reduction of −11.3 mmHg occurred 9 hours after administration, which was −8.3 mmHg lower than in the control group. The heart rate showed a tendency to be lower than that in the control group 3 hours after administration, but thereafter it showed an upward trend until 9 hours after administration. Thus, the blood pressure-lowering effect by oral administration of eggplants was confirmed.

The effective daily dose of AcCh administered to SHRs after 12 hours of fasting was 1.00×10⁻⁹ mol (0.146 μg)/kg, while the effective daily dose of AcCh administered to SHRs that did not fast in the repeated oral administration test was 1.00×10⁻⁸ mol (1.46 μg)/kg, at which a significant effect of suppressing the rise in blood pressure was observed (Non-Patent Document 21). This AcCh dose at which a blood pressure-lowering effect occurred in SHRs in normal condition upon oral ingestion was extrapolated to humans using Kleiber's law (Non-Patent Document 22). According to Kleiber's law, the mammalian metabolic rate is assumed to be proportional to the ¾ power of body weight, assuming the SHR weight to be 370 g and human weight to be 60 kg, the effective dose for humans is estimated to be 45.4 times the effective dose for SHRs. In other words, the effective daily intake of AcCh causing a blood pressure lowering effect is 3.70×10⁻⁹ mol (0.540 μg) in SHRs and 1.68×10−7 mol (24.5 μg) in humans.

Moreover, having already discovered the blood pressure-lowering effect and vasodilatory effect of fermented buckwheat (lactic acid-fermentation product of buckwheat plants), which comprises choline esters such as AcCh, BuCh, LaCh, PrCh as an active ingredient, the present inventor has made clear that a blood pressure-lowering effect is shown when purified AcCh, BuCh, PrCh are fed to SHRs by single oral administration (Patent Document 1 and 2). Further studies were conducted to examine the blood pressure-lowering effect of orally administered choline esters in humans.

Beverages comprising fermented buckwheat (containing 25 μg of choline ester per drink) were ingested on a daily basis for 4 weeks by persons with high-normal blood pressure and grade I hypertension (6 males, 6 females, 33 to 63 years old).

As a result, the subject's systolic blood pressure decreased by −11.8 mmHg (P=0.0080, t test) as compared to before ingestion. In other words, a significant decrease in blood pressure was observed in humans when a fermented buckwheat-containing beverage comprising 25 μg of choline esters as a blood pressure-lowering component was ingested continuously for 4 weeks. In this way, a blood pressure-lowering effect was confirmed at a dose very close to the effective AcCh dose extrapolated by Kleiber's law to humans from the dose causing a blood pressure-lowering effect in SHRs.

In the repeated oral administration test with SHR that did not fast, the blood pressure-lowering effect was greatly weakened at 2.00×10⁻⁹ mol (0.292 μg)/kg. This AcCh dose was extrapolated to humans using Kleiber's law (Non-Patent Document 22), and the lower limit of the daily effective dose for humans was determined at 3.36×10⁻⁸ mol (4.91 μg). In the same experiment, the blood pressure-lowering effect was greatly weakened at 2.00×10⁻⁷ mol (29.2 μg)/kg. When this AcCh dose was extrapolated to humans using Kleiber's law (Non-Patent Document 22), the upper limit of the daily human effective dose was 3.36×10⁻⁶ mol (491 μg). Therefore, the daily effective dose for humans with high blood pressure having genetic characteristics such as SHRs was estimated to be in the range of 5 to 500 μg.

The choline ester dose that causes the blood pressure lowering effect can in the end be determined by taking into consideration the human effective dose estimated as described above and by taking into consideration the amount of choline esters derived from dietary intakes other than the composition of the present invention.

### [Experiment 10] Single Oral Administration Test in Normotensive Rats Using an Eggplant Lyophilizate

By the same method as in Experiment 9, an eggplant lyophilizate was orally administered to Wistar-Kyoto rats (WKY rats), and the blood pressure lowering effect was examined. WKY rats are the parent stock of SHRs, and normotensive rats having genetic factors for acquiring high blood pressure were used as test controls. 14-week-old WKY male rats weighing 320 to 362 g were used as animals, and the same lyophilized eggplant powder as in Experiment 9 was used. The administered dose was as follows: 0.065 mg/kg (corresponding to 1.00×10−9 mol (0.146 μg)/kg of AcCh) and 6.5 mg/kg of lyophilized eggplant powder (corresponding to 1.00×10⁻⁷ mol (14.6 μg)/Kg of AcCh).

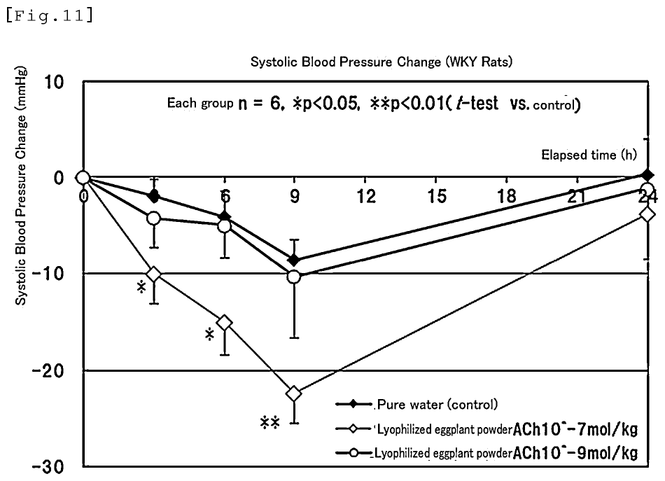

The results of the single oral administration test of lyophilized eggplant powder in WKY rats are shown in FIG. 11.

No change was observed in the blood pressure of WKY rats when a lyophilized eggplant powder corresponding to 1.00×10⁻⁹ mol/kg of AcCh was administered, which caused a blood pressure-lowering effect in SHRs. The administration of lyophilized eggplant powder corresponding to 1.00×10⁻⁷ mol/kg of AcCh caused the same blood pressure-lowering effect as lyophilized eggplant powder corresponding to 1.00×10⁻⁹ mol/kg of AcCh caused in SHRs. In other words, it has become clear that more lyophilized eggplant powder is required to cause blood pressure lowering action in WKY rats with normal blood pressure.

### [Experiment 11] Vascular Isometric Tension Measurement Test Using Ground Eggplant Samples

The ground eggplant samples used for the experiment were prepared as follows. A paste was prepared by washing a ‘Senshu’ water eggplant from Hannan-shi, Osaka-fu with water, by sterilizing the eggplant with hypochlorous acid, by cutting the edible part into slices with a width of 1 to 3 cm, and by grinding the slices with a commercially available food processor (Crush Millser IFM-C 20 G, Iwatani Sangyo Co., Ltd.). Moreover, a lyophilizate obtained by cutting an eggplant fruit into slices with a width of 1 to 3 cm and by heat sterilization at about 90° C. for about 5 minutes, or a hot air dried product of an eggplant fruit obtained by hot air drying at about 90° C. for about 1 to 2 hours, was ground into a powder by a mill mixer (Master, Tokyo Unicom Co., Ltd.). The ground samples were prepared by placing the product on a stainless steel sieve (JIS standard, 20 mesh), the product that passed was designated as 20 mesh-pass and the product that did not pass was designated as 20 mesh-on; Sample 1: ground product of fresh eggplant fruit 20 mesh-pass, Sample 2: ground product of fresh eggplant fruit 20 mesh-on, Sample 3: lyophilized powder 20 mesh-pass, Sample 4: lyophilized powder 20 mesh-on, Sample 5: hot air dried powder 20 mesh-pass; Sample 6: hot air dried powder 20 mesh-on.

Before the experiment, all samples were stored at −98° C. The choline ester content in Samples 1 and 2 was finely ground after lyophilisation and quantified by the methods described in “2. Extraction Method” and “3. LC-MS-MS Analysis”. Samples 3 and 5 were directly used as powder and quantified by the methods described in “2. Extraction Method” and “3. LC-MS-MS Analysis”. Samples 4 and 6 were finely ground and quantified by the methods described in “2. Extraction Method” and “3. LC-MS-MS Analysis”.

More than 99% of the choline esters in the ground eggplant samples were AcCh; the AcCh contents were as shown in Table 13.

TABLE 13
Acetylcholine Content in the Eggplant Samples

| | | Acetylcholine | |
|---|---|---|---|
| Sample | Ground Eggplant Sample | Content | |
| 1 | Ground product of fresh | 0.104 | mg/g FW |
| | eggplant fruit 20 mesh-pass | | |
| 2 | Ground product of fresh | 0.0786 | mg/g FW |
| | eggplant fruit 20 mesh-on | | |
| 3 | Lyophilized powder 20 mesh- | 2.36 | mg/g DW |
| | pass | | |
| 4 | Lyophilized powder 20 mesh-on | 1.65 | mg/g DW |
| 5 | Hot air dried powder 20 mesh- | 2.03 | mg/g DW |
| | pass | | |
| 6 | Hot air dried powder 20 mesh- | 1.49 | mg/g DW |
| | on | | |

The vascular isometric tension measurements were performed as follows.

14-week-old male SHRs (body weight 320 to 346 g) were put to death by laparotomy and exsanguination under ether anaesthesia, and the thoracic aorta was quickly removed. The isolated aorta was immersed in physiological saline and the blood was thoroughly rinsed off, and then the connective tissue and adipose tissue attached to the blood vessel were removed in a Krebs-Henseleit solution to prepare a ring specimen with a width of about 2 to 3 mm. The resulting ring specimens were attached inside an organ bus of an isometric tension test device (UFER UC-05A; Iwashiya Kishimoto Ika Sangyo K.K., Kyoto) filled with Krebs-Henseleit solution of 37° C. (119 mM NaCl/4.7 mM KCl/1.1 mM KH₂PO₄/1.2 mM MgSO₄/25 mM NaHCO₃, pH 7.4) in which a mixed gas of 95% O₂-5% CO₂ was aerated, then a static tension of 1.0 g was applied and the ring specimens were stabilized for 60 minutes. Thereafter, phenylephrine (PE, 0.3 μM) was added into the organ bus, AcCh (final concentration 100 μM) was added at the point where the blood vessels constricted and stabilized, and after it was confirmed that there was no problem in the endothelial state, the specimens were washed with a Krebs-Henseleit solution and returned to a static tension. After 15 minutes, the blood vessels were constricted again by the constriction agent PE (0.3 μM) to make sure that the constriction reached the maximum, and each of the ground eggplant samples was suspended in a Krebs-Henseleit solution so that the final AcCh concentration becomes 10⁻⁹, 10⁻⁸, 10⁻⁷, 10^{-6.5}, 10⁻⁶, 10^{-5.5}, 10⁻⁵ M. The change in tension was measured and recorded by using a transducer (UFER UM-20, Iwashiya Kishimoto Ika Sangyo Co., Ltd.), the dilation rate (%) was calculated by equation (1) hereinafter, and the mean value±standard error (Mean±S. E.) was determined by repeating the test results three times.

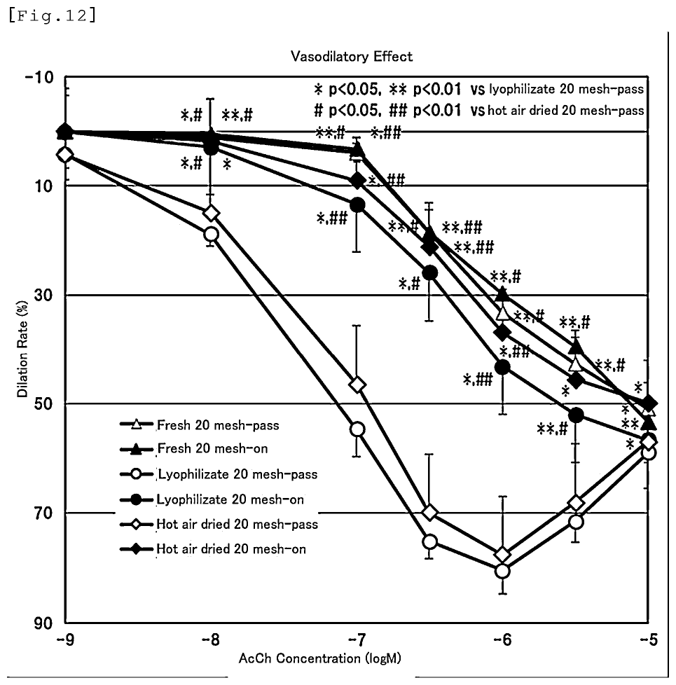

The vascular isometric tension test results of the ground eggplant samples are shown in FIG. 12, and the results, EC50 (50% effective concentration) determined by the probit method, and the results of the statistical analysis are shown in Table 14.

Dilation rate (%)={maximum tension (g)−tension when sample is added (g)}/maximum tension (g) Formula (1)

TABLE 14
Vascular Isometric Tension Test Results and EC₅₀(Mean ± Standard Error)

| | AcCh | | | | | | | | |
|---|---|---|---|---|---|---|---|---|---|
| Sample | Concentration (mol/L) | 10⁻⁹ | 10⁻⁸ | 10⁻⁷ | 10^{-6.5} | 10⁻⁶ | 10⁻ [symbol] ^{.} [symbol] | 10⁻ [symbol] | |
| 1 | Ground Eggplant | 1.4 × 10⁻ [symbol] | 1.4 × 10⁻² | 0.14 | 0.44 | 1.4 | 4.4 | 14 | EC₅₀ |
| | Concentration (mg/mL) | | | | | | | | (mg/mL) |
| | Dilation Rate (%) ± S.E. | 0 | 1.2 ± 0.38 | 3.9 ± 0.95 | 19 ± 4.1 | 33 ± 0.72 | 43 ± 2.9 | 51 ± 2.5 | 7.0 ± 0.63 |
| | p Value | 0.223 | 0.021 | 0.006 | 0.004 | 0.006 | 0.006 | 0.011 | 0.008 |
| | (t Test vs Sample 3) | | | | | | | | |
| | p Value | 0.082 | 0.037 | 0.014 | 0.018 | 0.013 | 0.041 | 0.241 | 0.008 |
| | (t Test vs Sample 5) | | | | | | | | |
| 2 | Ground Eggplant | 1.9 × 10⁻ [symbol] | 1.9 × 10⁻² | 0.19 | 0.59 | 1.9 | 5.9 | 19 | EC₅₀ |
| | Concentration (mg/mL) | | | | | | | | (mg/mL) |
| | Dilation Rate (%) ± S.E. | 0 | 0.57 ± 0.56 | 3.2 ± 1.7 | 19 ± 2.0 | 30 ± 1.1 | 39 ± 1.9 | 53 ± 1.6 | 9.9 ± 1.3 |
| | p Value | 0.223 | 0.009 | 0.016 | 0.006 | 0.005 | 0.007 | 0.003 | 0.017 |
| | (t Test vs Sample 3) | | | | | | | | |
| | p Value | 0.082 | 0.033 | 0.004 | 0.007 | 0.012 | 0.024 | 0.366 | 0.017 |
| | (t Test vs Sample 5) | | | | | | | | |
| 3 | Ground Eggplant | 6.2 × 10⁻ [symbol] | 6.2 × 10⁻⁴ | 6.2 × 10⁻ [symbol] | 2.0 × 10⁻² | 6.2 × 10⁻² | 0.20 | 0.62 | EC₅₀ |
| | Concentration (mg/mL) | | | | | | | | (mg/mL) |
| | Dilation Rate (%) ± S.E. | 4.2 ± 2.4 | 19 ± 2.2 | 55 ± 5.0 | 75 ± 3.3 | 80 ± 4.3 | 72 ± 3.9 | 59 ± 1.9 | 0.015 ± 0.0048 |
| | p Value | — | — | — | — | — | — | — | |
| | (t Test vs Sample 3) | | | | | | | | |
| | p Value | 0.986 | 0.310 | 0.461 | 0.558 | 0.771 | 0.675 | 0.607 | 0.476 |
| | (t Test vs Sample 5) | | | | | | | | |
| 4 | Ground Eggplant | 8.9 × 10⁻ [symbol] | 8.9 × 10⁻⁴ | 8.9 × 10⁻ [symbol] | 2.8 × 10⁻² | 8.9 × 10⁻² | 0.28 | 0.89 | EC₅₀ |
| | Concentration (mg/mL) | | | | | | | | (mg/mL) |
| | Dilation Rate (%) ± S.E. | 0.07 ± 0.035 | 2.9 ± 1.3 | 13 ± 2.1 | 26 ± 3.1 | 43 ± 1.2 | 52 ± 1.8 | 57 ± 1.9 | 0.24 ± 0.023 |
| | p Value | 0.226 | 0.015 | 0.025 | 0.0104 | 0.021 | 0.013 | 0.016 | 0.008 |
| | (t Test vs Sample 3) | | | | | | | | |
| | p Value | 0.080 | 0.012 | 0.007 | 0.013 | 0.008 | 0.078 | 0.913 | 0.014 |
| | (t Test vs Sample 5) | | | | | | | | |
| 5 | Ground Eggplant | 7.2 × 10⁻ [symbol] | 7.2 × 10⁻⁴ | 4.2 × 10⁻ [symbol] | 2.3 × 10⁻² | 7.2 × 10⁻² | 0.23 | 0.72 | EC₅₀ |
| | Concentration (mg/mL) | | | | | | | | (mg/mL) |
| | Dilation Rate (%) ± S.E. | 4.3 ± 1.3 | 15 ± 2.5 | 46 ± 4.2 | 70 ± 4.3 | 78 ± 4.3 | 68 ± 3.5 | 57 ± 1.8 | 0.027 ± 0.010 |
| | p Value | 0.986 | 0.310 | 0.461 | 0.558 | 0.771 | 0.675 | 0.607 | 0.476 |
| | (t Test vs Sample 3) | | | | | | | | |
| | p Value | — | — | — | — | — | — | — | |
| | (t Test vs Sample 5) | | | | | | | | |
| 6 | Ground Eggplant | 9.8 × 10⁻ [symbol] | 9.8 × 10⁻⁴ | 9.8 × 10⁻ [symbol] | 3.1 × 10⁻² | 9.8 × 10⁻² | 0.31 | 0.98 | EC₅₀ |
| | Concentration (mg/mL) | | | | | | | | (mg/mL) |
| | Dilation Rate (%) ± S.E. | 0 | 1.9 ± 0.94 | 9.0 ± 1.4 | 21 ± 2.2 | 37 ± 1.2 | 46 ± 2.1 | 50 ± 2.7 | 0.39 ± 0.050 |
| | p Value | 0.222 | 0.017 | 0.018 | 0.0098 | 0.015 | 0.006 | 0.044 | 0.017 |
| | (t Test vs Sample 3) | | | | | | | | |
| | p Value | 0.081 | 0.061 | 0.006 | 0.003 | 0.006 | 0.048 | 0.146 | 0.015 |
| | (t Test vs Sample 5) | | | | | | | | |

[symbol]

indicates data missing or illegible when filed

Table 14 shows the concentrations of the ground eggplant samples added so that the final AcCh concentration becomes 10⁻⁹, 10⁻⁸, 10⁻⁷, 10^{6.5}, 10⁻⁶, 10^{5.5}, 10⁻⁵ M; and since the lyophilized yield of ground fresh eggplant fruits used in this test was 6.0%, the ground eggplant concentration of the vascular isometric tension test of the ground product of fresh eggplant fruit 20 mesh-on was the highest with 19 mg/mL.

Chlorogenic acid and γ-aminobutyric acid (GABA) contents, which in addition to choline esters may also affect vasodilation, were 0.56 mg/g FW and 0.24 mg/g FW, respectively, and the concentration in this test system was 11 μg/mL and 4.8 μg/mL, respectively; at these concentrations, no effect on blood vessels was observed in the test using the respective standard samples. In test systems using other ground eggplants products, the contribution to the vasodilator effect was extremely limited because the concentration of these compounds was lower; therefore, it was considered that choline ester was the main vasodilator ingredient.

All the ground eggplant samples showed a dose-dependent vasodilatory effect. The lyophilized powder 20 mesh-pass (Sample 3), which exhibited the strongest vasodilator effect, showed a significant vasodilation (p<0.05) of 19%, compared to the time before the lyophilized eggplant powder was added, when a ground eggplant sample (6.2×10⁻⁴ mg/mL) was added so that the AcCh concentration in the chamber became 10⁻⁸ M, a significant dose-dependent vasodilation was shown until 10⁻⁶ M (ground eggplant sample 6.2×10⁻² mg/mL), and despite a slight constriction there was a significant vasodilation at higher concentrations than that. The maximum dilation rate was 80% when the ground eggplant sample (6.2×10⁻² mg/mL) was added to obtain an AcCh concentration of 10⁻⁶ M. The hot air dried powder 20 mesh-pass, which exhibited the second strongest vasodilatory effect, showed a significant vasodilation (p<0.05) of 15%, compared to the time before the lyophilized eggplant powder was added, when a ground eggplant sample (7.2×10⁻⁴ mg/mL) was added so that the AcCh concentration in the chamber became 10⁻⁸ M, a significant dose-dependent vasodilation was shown until 10⁻⁶ M (ground eggplant sample 7.2×10⁻² mg/mL), and despite a slight constriction there was a significant vasodilation at higher concentrations than that. The maximum dilation rate was 78% when the ground eggplant sample (7.2×10⁻² mg/mL) was added to obtain an AcCh concentration of 10⁻⁶ M. These results confirm the strong vasodilatory effect of dried eggplant powder.

On the other hand, Samples 4 and 6 show significant vasodilation (p<0.05) from an AcCh concentration of 10⁻⁷ M (lyophilized powder 8.9×10⁻³ mg/mL, hot air dried powder 9.8×10⁻³ mg/mL), and the maximum dilation rate was 57% for lyophilized powder and 50% for hot air dried powder when ground eggplant sample material was added so that the AcCh concentration became 10⁻⁵ M. When the vasodilatory effects of Sample 3 and Sample 4 were compared, a significantly strong vasodilatory effect was confirmed from an AcCh concentration of 10⁻⁸ M when Sample 3 was added, and the vasodilatory effect of 10⁻⁸ M was 6.5 times stronger than that of Sample 4. Similarly, from an AcCh concentration of 10⁻⁸ M, the vasodilatory effect of Sample 5 was significantly stronger than that of Sample 6, and the vasodilatory effect of 10⁻⁸ M was 7.8 times stronger. The above results confirm that Samples 3 and 5 have a stronger vasodilatory effect than Samples 4 and 6, respectively.

Moreover, Samples 1 and 2 showed significant vasodilation (p<0.05) from an AcCh concentration of 10⁻⁶ M (1.4 mg/mL) and 10^{-6.5} M (0.59 mg/mL), respectively; however, it has become clear that both have a weaker vasodilatory effect than dry powder, and that 20 mesh-pass dry powder exerts a high effect in vivo.

EC₅₀ is an index showing the strength of the biological effect of the test samples. In the main test of Sample 3, the vasodilatory effect EC₅₀ was 0.015 mg/mL and, except for Sample 5, a significantly stronger activity was confirmed than for other ground eggplants; the activity was 467 times stronger than for Sample 1, 660 times stronger than for Sample 2, 16 times stronger than for Sample 4 and 26 times stronger than for Sample 6. Furthermore, in Sample 5, which showed the second strongest vasodilatory effect, the EC₅₀ was 0.027 mg/mL and, except for Sample 3, a significantly stronger activity was confirmed than for other ground eggplants; the activity was 259 times stronger than for Sample 1, 367 times stronger than for Sample 2, 9 times stronger than for Sample 4 and 14 times stronger than for Sample 6.

From the above results, it was concluded that dried eggplant powder finer than 20 mesh had a strong vasodilatory effect.

### [Experiment 12] Repeated Oral Administration Test Using an Eggplant Lyophilizate

Eggplant lyophilizate was orally administered on a daily basis to spontaneously hypertensive rats (SHR) for 4 weeks to confirm its blood pressure-lowering effect and to evaluate the anti-stress effect by examining changes in urinary catecholamine levels.

After 6-week-old male SHRs (body weight 263 to 286 g) were conditioned for 6 days, they were divided into a control group (pure water administration, n=6) and a test group (lyophilized eggplant powder administration, n=6), they were separately reared using individual metabolic gauges under the conditions of an ambient temperature of 23±4° C., a humidity of 50±20%, and a 12-hour light and dark cycle (light period 5:30 to 17:30). The standard feed for breeding (MF, Oriental Yeast Co., Ltd.) was used as feed, and tap water was used as drinking water, both were freely consumed. Tosa-shi eggplants (produced in February, Kochi prefecture) were used as eggplant raw material, and a lyophilized eggplant powder was prepared by the method of [Experiment 9] above. The lyophilized eggplant powder dosage was 0.82 mg/kg (bw) per rat (AcCh equivalent: 10⁻⁸ mol/kg), and repeated oral administration war performed for 30 days using a stainless oral gastric tube. During the test period, food intake and water consumption were measured twice a week, body weight once a week, and urine output daily. The blood pressure (systolic phase, diastolic phase) was measured before the test and on day 7, 14, 21 and 28 after the start of the test in the same manner as in [Experiment 9] above. Each measurement was repeated three times, and the average value±standard error (Mean±S. E.) was determined.

Urinary catecholamine was quantified using a urine sample from the day before the blood pressure measurement. The collection of 24-hour urine samples was started on the day before the start of the repeated oral administration. The urine was collected once a day using a urine collection container to which 5N hydrochloric acid (1 mL) was added, and after measuring the amount of urine output, the obtained urine was stored frozen at −80° C. The samples were thawed for the analysis, the supernatant obtained by centrifugal separation (4° C., 1000×g, 3 minutes) was applied to a MonoSpin (Trade Mark) PBA (GL Sciences Co., Ltd.), and after washing, it was eluted with 1% aqueous acetic acid solution (400 μL), and the concentrate of the urinary catecholamine was used as a sample for analysis. The urinary catecholamine was quantified 3 times by using an HPLC system (Prominence HPLC System made by Shimadzu Corporation, Kyoto: system controller: CBM-20A, liquid delivery unit: LC-20AD, column oven: CTO-10A) and an electrochemical detector (ECD, GL Science Co., Ltd., ED 723 diamond electrode) using the conditions hereinafter, and the average value±standard error (Mean±S. E.) was determined.

### Column: Inertsil ODS-4 (4.6×250 mm, GL Science Co., Ltd.)

Flow rate: 0.8 mL/min, separation temperature: 35° C., standard injection amount: 20 μL
Mobile phase: acetate-citrate buffer/CH₃CN (100/16 v/v) Detection condition: ECD 800 mV (reference electrode Ag/AgCl)

As shown in Tables 15, 16 and 17, there were no significant differences in body weight, urine volume, water consumption and total food consumption in both groups throughout the feeding period.

TABLE 15
Daily Food Consumption and Water Consumption Change in Repeated Oral Administration Test (Mean ± Standard Error)

| Elapsed Days | | 0-3 | 4-7 | 8-10 | 11-14 | 15-17 | 18-21 | 22-24 | 25-28 |
|---|---|---|---|---|---|---|---|---|---|
| Ingested | Test Group | 19.1 ± 0.81 | 16.4 ± 0.32 | 21.0 ± 0.53 | 20.8 ± 0.56 | 20.0 ± 0.81 | 18.9 ± 0.28 | 21.4 ± 1.0 | 19.1 ± 0.27 |
| food | Control Group | 18.9 ± 0.59 | 16.3 ± 0.43 | 21.0 ± 0.58 | 19.7 ± 0.44 | 20.4 ± 0.66 | 18.8 ± 0.51 | 21.6 ± 0.96 | 17.5 ± 0.26 |
| | t test | 0.85 | 0.8 | 0.92 | 0.15 | 0.7 | 0.87 | 0.91 | 0.0021 |
| Ingested | Test Group | 27.1 ± 1.3 | 26.8 ± 1.1 | 31.1 ± 0.73 | 30.8 ± 1.2 | 30.3 ± 1.3 | 28.6 ± 0.91 | 29.2 ± 1.7 | 28.0 ± 0.89 |
| Water | Control Group | 28.3 ± 0.52 | 27.8 ± 1.1 | 32.0 ± 1.3 | 31.4 ± 1.7 | 32.1 ± 0.65 | 30.6 ± 1.4 | 33.2 ± 1.2 | 31.3 ± 1.7 |
| | t test | 0.4 | 0.52 | 0.56 | 0.79 | 0.23 | 0.25 | 0.079 | 0.11 |

TABLE 16
Change in Body Weight in Repeated Oral Administration
Test (Mean ± Standard Error)

| Elapsed Days | 0 | 7 | 14 | 21 | 28 |
|---|---|---|---|---|---|
| Test Group | 279.4 ± 4.6 | 296.8 ± 4.8 | 312.2 ± 4.3 | 326.6 ± 4.1 | 341.5 ± 4.2 |
| Control Group | 275.7 ± 4.9 | 294.4 ± 7.3 | 311.9 ± 7.6 | 326.1 ± 7.6 | 342.8 ± 9.4 |
| t test | 0.53 | 0.74 | 0.97 | 0.94 | 0.88 |

TABLE 17
Change in Urinary volume in Repeated Oral
Administration Test (Mean ± Standard Error)

| Elapsed Days | 0 | 6 | 13 | 20 | 27 |
|---|---|---|---|---|---|
| Test Group | 10.4 ± 0.67 | 11.3 ± 0.61 | 13.4 ± 0.75 | 11.7 ± 0.54 | 12.0 ± 0.40 |
| Control Group | 9.9 ± 0.63 | 13.2 ± 1.4 | 15.6 ± 1.4 | 12.5 ± 0.65 | 14.9 ± 1.4 |
| t test | 0.59 | 0.24 | 0.2 | 0.37 | 0.068 |

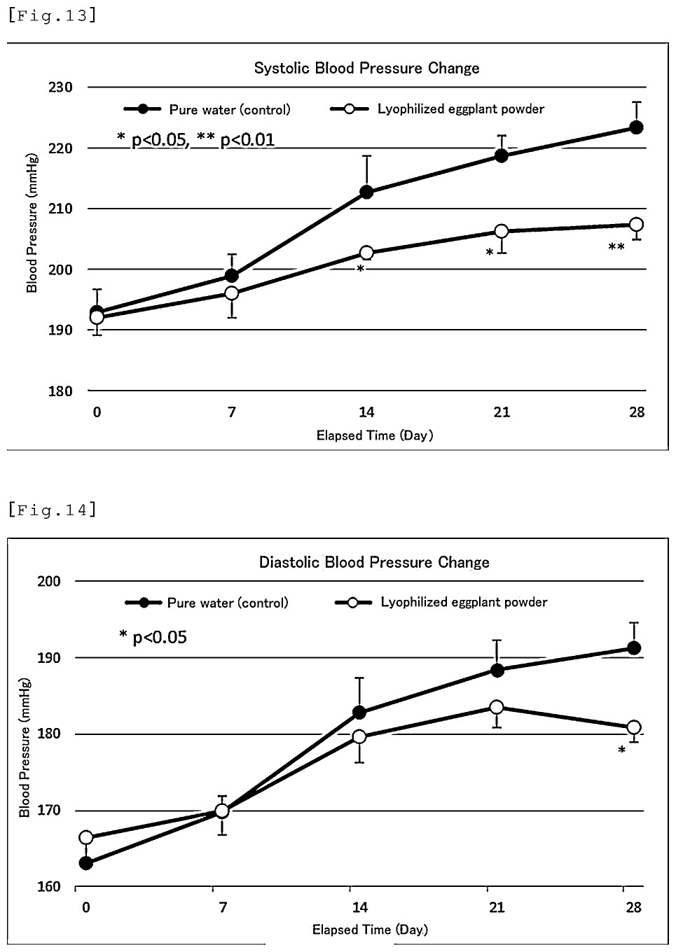

As shown in FIG. 13 and FIG. 14, the test group showed significantly lower systolic blood pressure on day 14, 21, 28 and significantly lower diastolic blood pressure on day 28 than the control group. The blood pressure-lowering effect due to lyophilized eggplant powder intake, which was revealed in the single oral administration test, was also confirmed in the repeated oral administration test.

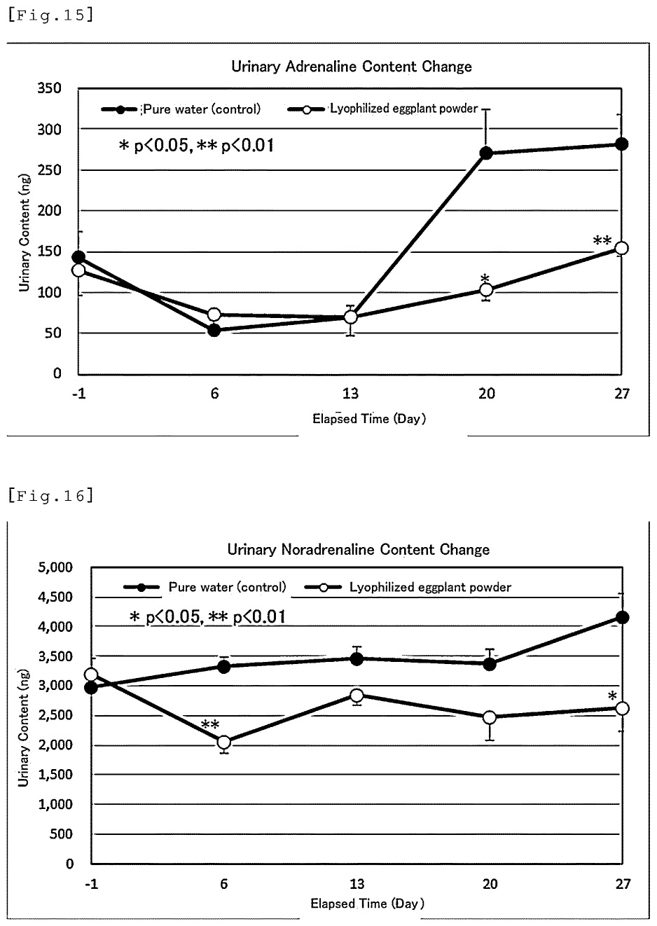

As shown in FIG. 15 and FIG. 16, the amount of adrenaline and noradrenaline which are typical catecholamines contained in urine was significantly lower in the test group than in the control group. On day 20 and day 27 after the start of the test, the amount of urinary adrenaline was significantly lower than in the control group, and on day 6 and day 27 after the start of the test the amount of urinary noradrenaline was significantly lower than in the control group.

Noradrenaline is a neurotransmitter released from sympathetic nerve terminals, and adrenalin is produced by converting noradrenaline in the adrenal gland. These catecholamines act on adrenergic receptors and are involved in the control of various organs and metabolic systems. The released catecholamines act on the effectors reached by nerves, and some are transferred to the blood to affect the whole body. Although catecholamines in blood are metabolized and inactivated, some are excreted into urine as they are. Therefore, the amount of catecholamine in urine is an indicator of sympathetic nerve activity in a living body. In other words, the amount of catecholamine in urine increases as a result of an increase in sympathetic nerve activity. The sympathetic nerves promote wakening, aggression, defense and escape behaviours. Catecholamine released by the enhancement of sympathetic nerve activity acts on the adrenergic alpha receptors of blood vessels, and causes the blood vessels to contract and the blood pressure to increase. Therefore, it is thought that catecholamine reduction due to the inhibition of sympathetic nerve activity is profoundly involved in the blood pressure-lowering mechanism of orally ingested choline esters.

Moreover, it is known that a living body enhances sympathetic nerve activity to cope with stress, and the amount of catecholamine increases due to stress. Therefore, catecholamines are known as stress indicators.

Catecholamine (adrenaline, noradrenaline, dopamine) is adopted as an indicator of the anti-stress effect in vivo of an anti-stress composition containing β-carotene as an active ingredient, and the anti-stress effect is shown by a significant reduction of catecholamine due to the composition intake (Patent Document 3). Moreover, the anti-stress effect is supported by significantly lower levels of blood adrenalin (described as epinephrine in the literature) and blood noradrenaline (described as norepinephrine in the literature) during restraint stress after administration of a yeast hydrolyzate (Non Patent Literature 23). In the evaluation of the anti-stress effect due to candesartan (angiotensin II type 1 receptor antagonist inhibitor), urinary catecholamine (adrenaline and noradrenaline) is used as an index of individual breeding stress in a metabolic cage, and each significant low value is considered to support the anti-stress effect.

In Experiment 12, the fact that the amount of urinary catecholamine was significantly reduced by the intake of lyophilized eggplant powder can be said to be because the intake of lyophilized eggplant powder inhibits sympathetic nerve activity and brings about an anti-stress effect. In other words, the anti-stress effect by intake of eggplant freeze-dried powder was observed. The vasodilatory effect of Experiment 11 was caused by the action of choline esters contained in eggplants on the muscarinic acetylcholine receptors (Non-patent Document 25). This anti-stress effect is also considered to be a result of the effect choline esters contained in eggplants have on muscarinic acetylcholine receptors to inhibit sympathetic nerve activity and to suppress catecholamine release from the nerve peripheral. Eggplant-based extracts and processed food containing a certain amount of choline ester are considered to have an anti-stress effect. The blood pressure-lowering effect and anti-stress effect caused by the inhibition of sympathetic nerve activity due to orally ingested choline esters are closely related to each other. Therefore, the difference in choline ester dose in SHRs and WKY rats in which a blood pressure-lowering effect was observed can also be adapted to the anti-stress effect.

### 4. Study of Implementation

According to the study of the present inventor, choline ester has been shown to have a blood pressure-lowering effect with a very small daily intake of 25 μg. The weight of agricultural products required for ingesting 25 μg of AcCh was set as the daily standard intake amount. The daily standard intake amount of 20 types of fresh agricultural products quantified in experiments 1 and 2 was calculated. The results are shown in Table 18.

TABLE 18
Daily Standard Intake Amount

| | Daily Standard |
|---|---|
| Crop Name | Intake Amount (g) |
| Lettuce | 7559 |
| Cabbage | 3675 |
| Cucumber | 3212 |
| Tomato | 3112 |
| Daikon Sprout | 2903 |
| Soybean | 1941 |
| Shishito Pepper | 1614 |
| Alfalfa Sprout | 1487 |
| Paprika | 1392 |
| Asparagus | 1227 |
| Apple | 1177 |
| Carrot | 1135 |
| Japanese Nashi Pear | 962 |
| Japanese Mountain | 872 |
| Yam | |
| Broccoli Sprout | 808 |
| Grape | 797 |
| Buckwheat Sprout | 597 |
| Green Pepper | 421 |
| Eggplant (Higomurasaki) | 0.41 |
| Bamboo Shoot | 0.25 |
| (Mosochiku) | |

As shown in Table 18, other than eggplants and bamboo shoots, 18 of the agricultural products require a very high intake in order to ingest 25 μg of AcCh. For example, with lettuce, it is necessary to ingest about 7.5 kg or more per day, which is not realistic as daily intake, and continuous intake on a daily basis is altogether impossible. On the other hand, eggplants and bamboo shoots can be expected to exhibit a blood pressure-lowering effect at an amount that allows continuous intake on a daily basis since the estimated daily intake amounts are only 0.41 g and 0.25 g respectively. Moreover, of the 7 varieties of eggplants used in this experiment, the 5 varieties ‘Senshu’ water eggplants, ‘Batten’ eggplants, ‘Koryo’ salad eggplants, ‘Higomurasaki’ eggplants and ‘Ryoma’ eggplants are characterized by soft skin, little bitterness, and can be eaten raw. When consumed raw, it is not necessary to take into consideration the decrease in choline esters due to cooking.

### INDUSTRIAL APPLICABILITY

The composition according to the present invention has a remarkable blood pressure-lowering effect, and by including this composition as an active ingredient, it is possible to produce functional display food or medicine for treating high blood pressure and the like.

1. A composition having a blood pressure-lowering effect and/or an anti-stress effect and comprising a choline ester as an active ingredient, wherein the composition is for oral ingestion and wherein the choline ester content is 5 μg to 50 mg.

2. The composition according to claim 1, wherein the composition is a food composition.

3. The composition according to claim 1, wherein the composition is a pharmaceutical composition for lowering blood pressure and/or for anti-stress.

4. The composition according to any one of claims 1 to 3, wherein the choline ester is derived from an edible plant.

5. The composition according to any one of claims 1 to 4, consisting of a lyophilized powder and/or a hot air-dried powder of edible plants.

6. The composition according to claim 5, consisting of a lyophilized powder and/or a hot air-dried powder of edible plants, which can pass through a 20 mesh sieve.

7. The composition according to any one of claims 1 to 4, wherein the composition is an extract obtained by extracting edible plants with ethanol or hydrous ethanol.

8. The composition according to any one of claims 4 to 7, wherein the edible plant is a fruit of a Solanaceae Solanum eggplant species (Solanum melongena) and/or a young shoot of a bamboo subfamily of Piceaceae (Poaceae, Bambusoideae, Bambuseae).

9. The composition according to any one of the claims 1 to 3, wherein the choline ester comprises one or more selected from the group consisting of acetylcholine, butyrylcholine and propionylcholine.

10. The composition according to claim 9, wherein the choline ester does not contain lactoylcholine.

11. The composition according to any one of claims 1 to 10, wherein the concentration of choline ester is 5 μg/g to 250 mg/g and the intake amount per day is adjusted from 5 μg to 50 mg.

12. The composition according to any one of claims 1 to 11, wherein the composition is frozen.

13. The composition according to any one of claims 1 to 3, wherein the composition is a part or the whole of the frozen fruit of a Solanaceae Solanum eggplant species (Solanum melongena).

14. A method of producing a composition for oral ingestion having a blood pressure-lowering effect and/or an anti-stress effect and comprising a choline ester as an active ingredient, wherein the method comprises
a step for making an edible plant into a lyophilized powder and/or a hot air-dried powder, and
a step for dispensing the lyophilized powder and/or the hot air-dried powder so that the choline ester content is 5 μg to 50 mg.

15. The method according to claim 14, wherein the edible plant is a fruit of a Solanaceae Solanum eggplant species (Solanum melongena) and/or a young shoot of a bamboo subfamily of Piceaceae (Poaceae, Bambusoideae, Bambuseae).

16. The method according to claim 14 or 15, wherein the method further comprises
a step for heating the edible plant.

17. The method according to any one of claims 14 to 16, wherein the method further comprises
a step for suspending the lyophilized powder and/or a hot air-dried powder of an edible plant in water, and a step for adding an acid to the suspension obtained.

18. The method according to claim 17, wherein the method further comprises
a step for adjusting the suspension to which acid has been added to a pH of 5.5 to 4.5.

19. A composition for oral ingestion having a blood pressure-lowering effect and/or an anti-stress effect and comprising a choline ester as an active ingredient, which is produced by a method according to any one of claims 14 to 18.

20. A method for producing an extract for oral ingestion having a blood pressure-lowering effect and/or an anti-stress effect and comprising a choline ester as an active ingredient, wherein the method comprises
a step for extracting an edible plant or a lyophilized powder and/or a hot air-dried powder of an edible plant with ethanol or hydrous ethanol.

21. The method according to claim 20, wherein the method comprises
a step for extracting an edible plant or a lyophilized powder and/or hot air-dried powder of an edible plant with ethanol.

22. The method according to claim 20, wherein the method comprises
a step for extracting an edible plant or a lyophilized powder and/or a hot air-dried powder of an edible plant with ethanol, and wherein the ethanol concentration of the hydrous ethanol is 25 to 60% (w/w), or 95% (w/w) or more.

23. The method according to any one of claims 20 to 22, wherein L-ascorbic acid is added to the ethanol or the hydrous ethanol used for the extraction.

24. The method according to any one claims 20 to 23, wherein the method comprises
a step for adjusting the choline ester content of the extract from 5 μg to 50 mg.

25. An extract for oral ingestion having a blood pressure-lowering effect and/or an anti-stress effect and comprising a choline ester as an active ingredient produced according to any one of claims 20 to 24.
